(19) United States
(12) Patent Application Publication
(10) Pub. No.: US 2009/0182131 A1
(43) Pub. Date: Jul. 16, 2009

(54) Methods for removing anti-MHC antibodies from a sample

(76) Inventors: William H. Hildebrand, Edmond, OK (US); Rico Buchli, Edmond, OK (US)

Correspondence Address: DUNLAP CODDING, P.C., PO BOX 16370, OKLAHOMA CITY, OK, 73113, US

(21) Appl. No.: 12/002,005

(22) Filed: Dec. 14, 2007

Related U.S. Application Data
(62) Division of application No. 10/669,925, filed on Sep. 24, 2003.
Application No. 10/669,925 is a continuation-in-part of application No. 10/337,161, filed on Jan. 2, 2003.
Application No. 10/337,161 is a continuation-in-part of application No. 10/022,066, filed on Dec. 18, 2001, now abandoned.
(60) Provisional application No. 60/413,842, filed on Sep. 24, 2002.
(60) Provisional application No. 60/474,655, filed on May 30, 2003.
(60) Provisional application No. 60/347,906, filed on Jan. 2, 2002.

(51) Int. Cl. C07K 1/14

(52) U.S. Cl. 530/413

(57) ABSTRACT

The present invention relates generally to anti-MHC assay methodologies utilizing functionally active, recombinantly produced, and truncated individual soluble MHC trimolecular complexes that are linked to a substrate. The methods include reacting a sample with the substrate having the MHC trimolecular complex linked thereto, whereby antibodies specific for the at least one MHC trimolecular complex linked to the substrate are removed from the biological sample.

### CROSS-REFERENCE TO RELATED APPLICATIONS

This application is a divisional of U.S. Ser. No. 10/669,925, filed Sep. 24, 2003; which claims benefit under 35 U.S.C. 119(e) of provisional applications U.S. Ser. No. 60/413,842, filed Sep. 24, 2002, and U.S. Ser. No. 60/474,655, filed May 30, 2003.

Said application U.S. Ser. No. 10/669,925 is also a continuation-in-part of U.S. Ser. No. 10/337,161, filed Jan. 2, 2003; which claims benefit under 35 U.S.C. 119(e) of U.S. Ser. No. 60/347,906, filed Jan. 2, 2002. Said U.S. Ser. No. 10/337,161 is also a continuation-in-part of U.S. Ser. No. 10/022,066, filed Dec. 18, 2001, the contents of which are hereby expressly incorporated herein by reference in their entirety.

Each of the above-referenced patents and patent applications are hereby expressly incorporated herein by reference in their entirety.

### STATEMENT REGARDING FEDERALLY SPONSORED RESEARCH OR DEVELOPMENT

Not Applicable.

### BACKGROUND OF THE INVENTION

1. Field of the Invention

The present invention relates generally to the utilization of functionally active, individual soluble HLA molecules that are isolated and purified substantially away from other proteins to identify antibodies specific for a specific purified functionally active HLA molecule. Anti-HLA antibodies are a contraindication for clinical transplantation, and the provision of individual HLA molecules facilitates the optimal allogeneic transplantation of organs, tissue, and bone marrow through the unambiguous identification of anti-HLA antibodies.

2. Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules, which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often have deleterious or even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens. The examination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively develop therapies and diagnostics for transplantation, autoimmune disorders, and for vaccine development.

In recent years, some progress has been made, in the screening of allo-antibodies in human sera. But success in unraveling patterns of allo-antibody recognition of HLA has been impeded by method-specific issues such as the multiple-specificity approach, the fact that many HLA molecules exist, the observation that one antibody can recognize multiple HLA molecules, and the fact that one individual can have antibodies against multiple HLA molecules. Primarily, the production and isolation of individual native HLA molecules is laborious, low-throughput, and difficult to reproduce. Patterns of antibody recognition to HLA are therefore limited to screening antibodies against a mixture of HLA molecules.

A major disadvantage in these assays is the availability of pure, single specificity molecules. For researchers and clinicians world wide, a major impediment for MHC Class I studies has been the difficulty of isolating sufficiently large quantities of Class I molecules from mammalian tissue culture cells [Bjorkman et al., 1987]. Several attempts have been made to express high levels of Class I molecules in bacteria [Dedier et al, 2001; Garboczi et al., 1992; Parker et al., 1992; and Silver et al., 1991] and insect cells [Levy et al., 1990]. However, none of these reported systems seems particularly useful in generating Class I molecules for sera screening and are still not considered to be a breakthrough since they only inefficiently promote heavy chain β2m heterodimer formation. The purification of native Class I molecules from mammalian cells requires time-consuming and cumbersome methods and does not deliver sufficient quantities; and native molecules from mammalian cells typically consist of a mixture of different HLA molecules which is not applicable in single specificity studies. A primary advantage of producing HLA molecules in mammalian cells is that they are naturally loaded with endogenous peptides. Such natural loading with thousands of different endogenous peptides ensures that the many different anti-HLA allo-antibodies that arise are able to detect the different configurations of an HLA molecule that arise from the binding of different peptides (Solheim and Bluestone). In essence, the peptide loaded will influence antibody reactivity, and production of native individual HLA in mammalian cells facilitates natural antibody recognition as influenced by peptide loading.

One example of an assay to determine the presence of antibodies or other receptors specific for alloantigens is described in U.S. Pat. No. 5,482,841, issued to Buelow on Jan. 9, 1996, the contents of which are hereby expressly incorporated herein by reference. Buelow discloses extracting HLA from a cellular source with a mild detergent and partially purifying by precipitation of potentially interfering components, and then using such extracted HLA in a sandwich assay to determine the presence of receptors specific for the HLA extracted from the cells. However, the HLA extracted by the methods of Buelow are a mixture of HLA molecules, and such mixture is neither characterized nor separated, so that the identity and specificity of the HLA molecules are not determined. At best, potential donor:recipient pairs can be tested to determine whether the recipient harbors antibodies to the HLA present on the donor's cells. However, it is not feasible to test every single potential donor:recipient pair, and the anti-HLA antibodies present in the recipient need to be identified so that further screening of potential donors carrying such HLAs can be eliminated. In addition, detergent solubilization yields low amounts of HLA, and the lysis of the entire cell to obtain HLA introduces all cellular proteins, as well as lipids and other cell components, which means additional protein purification and loss of HLA. Therefore, methods are desirable in which the specificity of anti-HLA antibodies present in recipients can be easily and positively determined, and such methods would require the use of individual isolated, native and purified HLA molecules.

However, prior to the presently claimed and disclosed invention there has been no readily available source of individual isolated and purified HLA molecules. The quantities of HLA protein previously available have been small and typically consist of a mixture of different HLA molecules in a detergent lysate. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. In addition, detergent cell lysis requires killing the cell producing the HLA, and in essence reducing the amount of HLA obtained from any given cell to that HLA on the cell surface. Therefore, prior to the presently claimed and disclosed invention(s), a need existed in the art for a method of producing substantial quantities of individual HLA class I or class II molecules so that they can be readily purified and isolated independent of other HLA class I or class II molecules and utilized in methods of identifying anti-HLA antibodies specific for individual HLA molecules. Such individual isolated and purified HLA molecules, when provided in sufficient quantity and purity as described herein, provide a powerful tool for antibody screening.

Therefore, there exists a need in the art for improved methods of identifying anti-HLA antibodies in a sample. In one exemplary embodiment, the present invention solves this need by (1) providing methods for isolating and purifying substantial quantities of individual HLA molecules substantially away from other proteins; (2) coupling the production and purification of soluble HLA molecules with assay methodology that involves capturing the individual, soluble HLA molecules on a substrate; (3) reacting captured soluble HLA molecules with a sample containing anti-HLA antibodies; and finally (4) detecting the presence of anti-HLA antibodies in the sample by visualizing binding of the anti-HLA antibodies to the individual soluble HLA molecules.

### SUMMARY OF THE INVENTION

The present invention is directed to anti-HLA assay methodology that utilizes a functionally active, individual soluble HLA molecule purified substantially away from other proteins such that the individual soluble HLA molecule maintains the physical, functional and antigenic integrity of the native HLA molecule. The term “physical, functional and antigenic integrity of the native HLA molecule”, as used herein, will be understood to mean that the soluble HLA molecules exhibit the same structure (including primary, secondary, tertiary and quaternary) as the extracellular portion of the native HLA molecules, that they are identical in functional properties to an HLA molecule expressed from the HLA allele mRNA or gDNA and thereby bind peptide ligands in an identical manner as full-length, cell-surface-expressed HLA molecules, and that they are recognized by the cellular machinery responsible for responses to specific HLA-peptide complexes, that is NK and T cells.

The functionally active, individual soluble HLA molecule is a Class I HLA molecule or a Class II HLA molecule, and may have an endogenous peptide loaded therein.

The functionally active, individual soluble HLA molecules may be produced by several methods, including but not limited to the following. In one embodiment, HLA allele mRNA from a source is isolated and reverse transcribed to obtain allelic cDNA. In a separate embodiment, gDNA encoding a HLA allele is obtained. The allelic cDNA or gDNA is amplified by PCR utilizing at least one class I specific primer that truncates the allelic cDNA or gDNA, thereby resulting in a truncated PCR product having the coding regions encoding cytoplasmic and transmembrane domains of the allelic cDNA removed such that the truncated PCR product has a coding region encoding a soluble HLA molecule. The at least one class I specific primer may include a stop codon incorporated into a 3′ primer, or the at least one class I specific primer may include a sequence encoding a tail such that the soluble HLA molecule encoded by the truncated PCR product contains a tail attached thereto that facilitates in purification of the soluble HLA molecules produced therefrom, as well as facilitates capturing of the soluble HLA molecules produced therefrom on a substrate for use in the anti-HLA assay.

The truncated PCR product is then inserted into a mammalian expression vector to form a plasmid containing the truncated PCR product having the coding region encoding a soluble HLA molecule, and the plasmid is electroporated or transfected into at least one suitable host cell. The mammalian expression vector contains a promoter that facilitates increased expression of the truncated PCR product. The host cell may lack expression of Class I HLA molecules.

A hollow fiber bioreactor unit is inoculated with the at least one suitable host cell containing the plasmid containing the truncated PCR product such that the hollow fiber bioreactor unit produces soluble HLA molecules, wherein the soluble HLA molecules are folded naturally and are trafficked through the cell in such a way that they are identical in functional properties to an HLA molecule expressed from the HLA allele mRNA and thereby bind peptide ligands in an identical manner as full-length, cell-surface-expressed HLA molecules. The individual, soluble HLA molecules are then harvested from the hollow fiber bioreactor unit and purified substantially away from other proteins. The purification process involves affinity column purification and filtration. The purified individual soluble HLA molecules maintain the physical, functional and antigenic integrity of the native HLA molecule.

When HLA allele mRNA is used, the source is selected from the group consisting of mammalian DNA and an immortalized cell line. When gDNA which encodes an HLA allele is used, the gDNA is obtained from blood, saliva, hair, semen, or sweat.

The present invention is directed to an assay for detecting the presence of anti-HLA antibodies in a sample. The assay includes a substrate, such as a solid support, a functionally active, individual soluble HLA molecule linked directly or indirectly to the substrate, and a means for detecting an anti-HLA antibody bound to the functionally active, individual soluble HLA molecule. The functionally active, individual soluble HLA molecule has been purified substantially away from other protein such that the individual soluble HLA molecule maintains the physical, functional and antigenic integrity of the native HLA molecule. The means for detecting an anti-HLA antibody may be a labeled antibody that recognizes at least one of anti-human IgG, IgM and IgA antibodies.

The substrate may be a solid support, such as a well, a bead (such as a flow cytometry bead), a membrane, an ELISA plate, a matrix, or combinations thereof. The individual, soluble HLA molecule may be indirectly attached to the substrate via an anchoring moiety. One embodiment of an anchoring moiety that may be used in accordance with the present invention is an antibody to the individual, soluble HLA molecule, such as but not limited to, W6/32, anti-beta 2m, other Pan specific HLA class I antibodies, or combinations thereof. Another embodiment of an anchoring moiety that may be used in accordance with the present invention is an affinity reagent that binds to a tail or tag attached to the individual, soluble HLA molecule. For example, the individual soluble HLA molecule may have a histidine tag attached thereto, and the affinity reagent may be nickel and/or copper. In another example, the individual soluble HLA molecule may have a biotinylation signal attached thereto, and the affinity reagent utilized therewith is avidin or streptavidin. In a further alternative, the individual, soluble HLA molecule may be provided with a VLDLr or FLAG tail, and an antibody that recognizes VLDLr or FLAG may be utilized as the affinity reagent.

The present invention also includes a method for detecting the presence of anti-HLA antibodies in a sample. The method includes providing the assay described herein above and reacting a sample with the substrate having the functionally active, individual soluble HLA molecule linked thereto. The substrate is then washed to remove unbound portions of the sample, and the substrate having the functionally active, individual soluble HLA molecule linked thereto is reacted with the means for detecting anti-HLA antibodies. The final step of the method includes determining that anti-HLA antibodies specific for the native HLA molecule are present in the sample is the means for detecting anti-HLA antibodies is positive.

The present invention also includes a kit that includes the assay described herein above in addition to at least one control sample selected from the group consisting of a positive control sample comprising anti-HLA antibodies that bind to the functionally active, individual soluble HLA molecule, a negative control sample wherein no anti-HLA antibodies that bind to the functionally active, individual soluble HLA molecule are present, and combinations thereof.

### BRIEF DESCRIPTION OF THE DRAWINGS

FIG. 1 is a graphical representation of a Class I location and sHLA class I construction strategy. (A) Simple map of the human MHC region with the class I HLA-B, -C, and -A loci noted. Genetic distances are in kilobases. (B) The basic exon structure of HLA class I gene transcripts. Seven exons encode the class I heavy chain. (C) PCR strategy for truncating the class I molecule so that it is secreted rather than surface bound.

FIG. 2 is a pictorial representation of native and recombined truncated form of sHLA which differ in the presence of a transmembrane and cytosolic region in the native molecule. Both forms show no differences in their ambiguity and peptide presenting properties.

FIG. 3 is a three dimensional pictorial representation of a truncated molecule. The top view is visualizing the α₁ and α₂ domains harboring the peptide. The side view shows the full molecule with a detailed view of α3 and β2m domains.

FIG. 4 is a pictorial representation showing the peptide binding platform in more detail where two α helices form the rim and seven β sheets form the bottom of the binding groove.

FIG. 5 is a graphical representation of a column-loading profile of the sHLA class I molecule B*0702BSP visualized by an ELISA procedure, demonstrating that the W6/32-coupled affinity column can be saturated with crude harvest containing sHLA-B*0702BSP.

FIG. 6 is a graphical representation of the washing step for the W6/32-coupled affinity column of FIG. 5 visualized by spectrophotometry as well as an ELISA procedure. FIG. 6 also includes an SDS PAGE gel that shows selected wash fractions containing proteins that do not correspond to the class I heavy chain (HC) and β2m light chain and are being removed from the column. After wash fraction 20, no contaminating protein is present.

FIG. 7 is a graphical representation of the elution pattern of sHLA-B*0702BSP from the W6/32-coupled affinity column of FIG. 5 visualized by spectrophotometry as well as an ELISA procedure. FIG. 7 also includes an SDS PAGE gel that shows fractions containing protein that correspond to the class I heavy chain (HC) and β2m light chain.

FIG. 8 is a pictorial representation illustrating the Protein Sequence Data for MHC Class I-HLA-A*0201T.

FIG. 9 is a tabular representation illustrating the amino acid analysis of B*1512 following proteolysis of the whole molecule.

FIG. 10 is a graphical representation showing Superdex™ chromatography to demonstrate sample purity of sHLA-B*1512T.

FIG. 11 is a graphical representation illustrating a Triple analysis of B*1512T. It shows a separation of sHLA under denaturing and under native conditions.

FIG. 12 is a pictorial representation of an SDS-PAGE gel analysis of several purified sHLA samples confirming the purity with this procedure.

FIG. 13 is a pictorial representation of a Western blot analysis to follow the HC and β2m subunits of sHLA with subunit-specific antibodies.

FIG. 14 is a pictorial representation illustrating anti-calreticulin blot of full-length HLA-B27 (+), HLA negative cell line 721.221 (−) and various constructs of soluble HLA-B15 molecules immunoprecipitated with the HLA-specific antibody HC-10.

FIG. 15 is a pictorial representation depicting a motif comparison between sHLA-B*1501 and membrane bound B*1501 from another laboratory.

FIG. 16 is a pictorial representation showing a fluorescence polarization scheme allowing the detection of bound and free peptides to the sHLA complex in solution without separation using radiometric measurements of parallel and perpendicular fluorescent intensities. Free peptides create a low FP signal where bound peptides show high FP values.

FIG. 17 (A-D) are graphical representations showing (A) the association of peptide P2(A*0201), (B) the dose-response of the reaction in view of sHLA concentration as well as (C) peptide concentration, and (D) the determination of the affinity to confirm structural integrity of the sHLA complex A*0201T used.

FIG. 18 is a graphical representation summarizing the purification and characterization procedures for soluble human HLA proteins of the present invention.

FIG. 19 is a graphical representation summarizing the sandwich assay of the present invention.

FIG. 20 is a chart showing the activity confirmation of sHLA B*1512T using a gradient of sHLA concentrations directly coated to an ELISA plate.

FIG. 21 is a chart showing reactivity of sHLA A*0201T directly coupled to beads.

FIG. 22 is a chart showing One Lambda A2/A28 antibody-reactivity against a selection of sHLA molecules of the present invention, tested using two different capturing methods.

FIG. 23 is a chart showing One Lambda B12 antibody-reactivity against a selection of sHLA molecules of the present invention, tested using two different capturing methods.

FIG. 24 is a chart illustrating sera screen ELISA of HLA A, B and C alleles using W6/32 and anti-β2m capturing systems.

FIGS. 25-26 are charts showing Bw4/Bw6 reactivity of sHLA molecules.

FIG. 27 is a chart illustrating an ELISA procedure to test capturing efficiency of W6/32 and TP25.99 antibodies, using HC10 and secondary antibody as negative controls.

FIG. 28 are charts illustrating screening results for sera OF1414, JH-9B, JH-PAGE, JH-Watson, JH-1I, and JH-Taylor using the sera screen ELISA prototype of the present invention.

FIG. 29 is a chart illustrating positive and negative serum reaction against sHLA A*0201T allele after background subtraction detected using the sandwich ELISA technique with W6/32 as capturing antibody and anti-human IgG(HRP) as secondary antibody.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The present invention combines assay methodologies for identifying anti-HLA antibodies specific for particular HLA molecules with novel and non-obvious methodologies for the production, isolation and purification of individual, soluble MHC molecules substantially away from other proteins. The method of production of individual, soluble MHC molecules has previously been described in detail in parent application U.S. Ser. No. 10/022,066, filed Dec. 18, 2001, entitled “METHOD AND APPARATUS FOR THE PRODUCTION OF SOLUBLE MHC ANTIGENS AND USES THEREOF,” the contents of which are hereby expressly incorporated in their entirety by reference herein. A brief description of this methodology is included herein below for the purpose of exemplification and should not be considered as limiting. One of ordinary skill in the art, given the disclosure in the 10/022,066 application would be truly capable of producing individual soluble MHC molecules to be used with the presently disclosed and claimed isolation and purification methodologies. It should be preliminary noted, however, that the presently claimed and disclosed isolation and purification methodologies can be used with HLA molecules (soluble or non-soluble, membrane bound or non-membrane bound) obtained by any means and should not be regarded as being limited to soluble HLA molecules produced according to the methodologies claimed and disclosed in the 10/022,066 application. In the event HLA molecules produced according to methodologies other than those produced according to methodologies disclosed and claimed in the 10/022,066 application are used in the isolation and purification methodologies disclosed and claimed herein, one of ordinary skill in the art (given in the present specification, drawings and claims) would be capable of making any necessary modifications or derivations to such HLA molecules such that they may be used in the isolation and purification methodologies presently claimed and disclosed herein in an efficient and accurate manner.

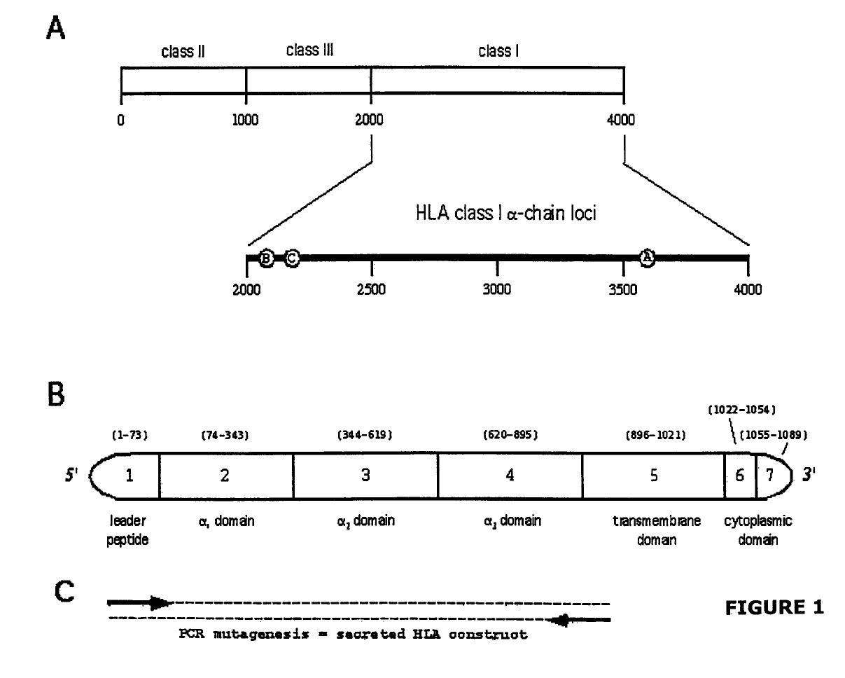

### DETAILED DESCRIPTION OF FIGS. 1-29

### Exemplary Production of Individual, Soluble MHC Molecules

The methods of the present invention may, in one embodiment, utilize a method of producing MHC molecules (from genomic DNA or cDNA) that are secreted from mammalian cells in a bioreactor unit. Substantial quantities of individual MHC molecules may be obtained in the manner by more particularly modifying class I or class II MHC molecules so that they are capable of being secreted, isolated, and purified. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this manner, the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity is assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

While hollow fiber bioreactor units or CELL PHARM®s have been described herein for utilization in the culturing methods of the present invention, it is to be understood that any large scale mammalian tissue culture system evident to a person having ordinary skill in the art may be utilized in the methods of the present invention, and therefore the present invention is not specifically limited to the use of a hollow fiber bioreactor unit or a CELL PHARM®.

The method of producing MHC molecules utilized in the present invention and described in detail in parent application U.S. Ser. No. 10/022,066 begins by obtaining genomic or complementary DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In one embodiment a nested or hemi-nested PCR is applied to produce a truncated form of the class I or class II gene so that it will be secreted rather than anchored to the cell surface. FIG. 1 illustrates the PCR products resulting from such nested PCR reactions. In another embodiment the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated clones of the desired MHC molecule are then transfected into a mammalian cell line. When such cell line is transfected with a vector encoding a recombinant class I molecule, such cell line may either lack endogenous class I MHC molecule expression or express endogenous class I MHC molecules.

One of ordinary skill in the art would note the importance, given the present invention, that cells expressing endogenous class I MHC molecules may spontaneously release MHC into solution upon natural cell death. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a HIS tail may be attached to the protein by the PCR reaction or may be encoded by the vector into which the PCR fragment is cloned, and such HIS tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified. Examples of other tags that may be utilized in accordance with the present invention include, but are not limited to, VLDLr, FLAG, BSP and the like.

Cloned genomic DNA fragments contain both exons and introns as well as other non-translated regions at the 5′ and 3′ termini of the gene. Following transfection into a cell line which transcribes the genomic DNA (gDNA) into RNA cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein. Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the present invention provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, the present invention which starts with MHC genomic DNA clones allows for the production of MHC in cells from various species.

A key advantage of starting from gDNA is that viable cells containing the MHC molecule of interest are not needed. Since all individuals in the population have a different MHC repertoire, one would need to search more than 500,000 individuals to find someone with the same MHC complement as a desired individual—such a practical example of this principle is observed when trying to find a donor to match a recipient for bone marrow transplantation. Thus, if it is desired to produce a particular MHC molecule for use in an experiment or diagnostic, a person or cell expressing the MHC allele of interest would first need to be identified. Alternatively, in the method of the present invention, only a saliva sample, a hair root, an old freezer sample, or less than a milliliter (0.2 ml) of blood would be required to isolate the gDNA. Then, starting from gDNA, the MHC molecule of interest could be obtained via a gDNA clone as described herein, and following transfection of such clone into mammalian cells, the desired protein could be produced directly in mammalian cells or from cDNA in several species of cells using the methods described herein.

Current experiments to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not as easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the present invention show that >5 milliliters of blood that is less than 3 days old is required to obtain sufficient RNA for MHC cDNA synthesis. Thus, by starting with gDNA, the breadth of MHC molecules that can be readily produced is expanded. This is a key factor in a system as polymorphic as the MHC system; hundreds of MHC molecules exist, and not all MHC molecules are readily available. This is especially true of MHC molecules unique to isolated populations or of MHC molecules unique to ethnic minorities. Starting class I or class II MHC molecule expression from the point of genomic DNA simplifies the isolation of the gene of interest and insures a more equitable means of producing MHC molecules for study; otherwise, one would be left to determine whose MHC molecules are chosen and not chosen for study, as well as to determine which ethnic population from which fresh samples cannot be obtained and therefore should not have their MHC molecules included in a diagnostic assay.

While cDNA may be substituted for genomic DNA as the starting material, production of cDNA for each of the desired HLA class I types will require hundreds of different, HLA typed, viable cell lines, each expressing a different HLA class I type. Alternatively, fresh samples are required from individuals with the various desired MHC types. The use of genomic DNA as the starting material allows for the production of clones for many HLA molecules from a single genomic DNA sequence, as the amplification process can be manipulated to mimic recombinatorial and gene conversion events. Several mutagenesis strategies exist whereby a given class I gDNA clone could be modified at either the level of gDNA or at the cDNA resulting from this gDNA clone. The process of producing MHC molecules utilized in the present invention does not require viable cells, and therefore the degradation which plagues RNA is not a problem.

### Purification of Individual, Soluble MHC Molecules

The ability to produce large quantities of single specificity sHLA molecules allows for assay procedures to be quantitative and resistant to interferences encountered in biological matrices as well as also being reliable, highly reproducible, sensitive, and therefore applicable for high-throughput systems. Alternative economical methodologies for obtaining large quantities of sHLA molecules do not currently exist since: (1) there is no readily available source of individual HLA molecules; (2) purification of native class I molecules from mammalian cells requires time-consuming and cumbersome purification methods and does not deliver sufficient quantities; and (3) native molecules from mammalian cells typically consist of a mixture of different HLA molecules. Such a mixture of specificities is not useful and/or applicable for single specificity studies.

HLA class I molecules are antigen-presenting glycoproteins expressed universally in nucleated cells. In humans, heavy chains are encoded at 3 loci (B, C, and A) within the MHC on the short arm of chromosome 6 (FIG. 1A). FIG. 1B illustrates each α-chain comprised of α₁, α₂, and α₃ domains, as well as a transmembrane domain, which tethers the molecule to the cell surface and a short C-terminal cytoplasmic domain. In contrast, the light chain is encoded outside of the MHC (on chromosome 15 in humans) and bears no such anchoring domain; it instead associates noncovalently with the α₃ domain of the heavy chain. FIG. 1C illustrates the approach for creating sHLA class I transcripts. The PCR primers truncate the class I heavy chain following exon 4, just before the transmembrane domain and cytoplasmic domains. Using this PCR truncation strategy, we have successfully created sHLA class I gene products for a series of fifty divergent HLA-molecules. Class I sHLA gene constructs created as in FIG. 1C are cloned and DNA sequenced to insure fidelity of each clone. The individual class I constructs are then subcloned into a suitable protein expression vector.

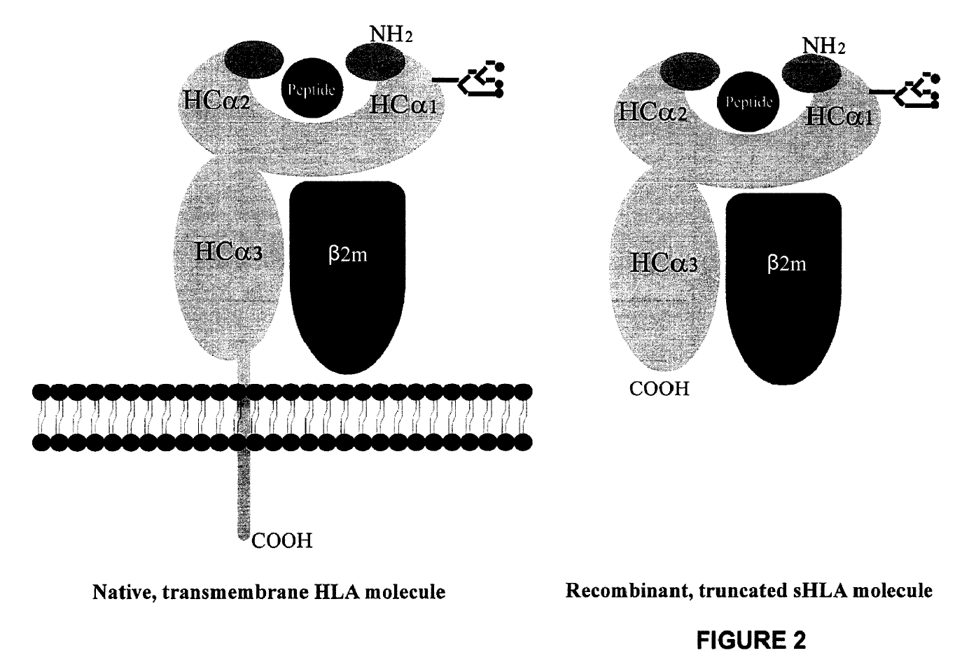

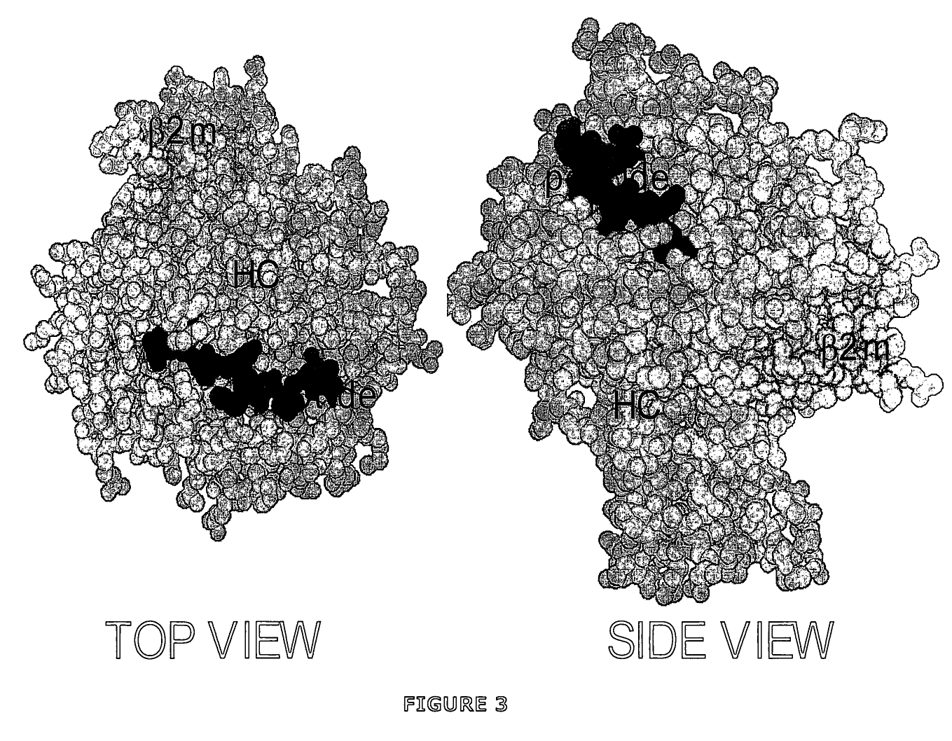

Produced in transfected B cells, sHLA molecules have close to identical primary structures as papain solubilized HLAs. Truncated molecules have been shown by the present inventors to maintain their structural integrity. In addition, HLA-Aw68, from which the complete alpha 3 domain has been proteolytically removed, shows no gross morphological changes compared to the intact protein. A decameric peptide complexed with the intact HLA-Aw68 is seen to bind to the proteolized molecule in the conventional manner, demonstrating that the alpha 3 domain is not required for the structural integrity of the molecule or for peptide binding. Pictures of sHLA graphics (FIG. 2) and 3D structures (FIG. 3) more clearly visualize how the molecules look like.

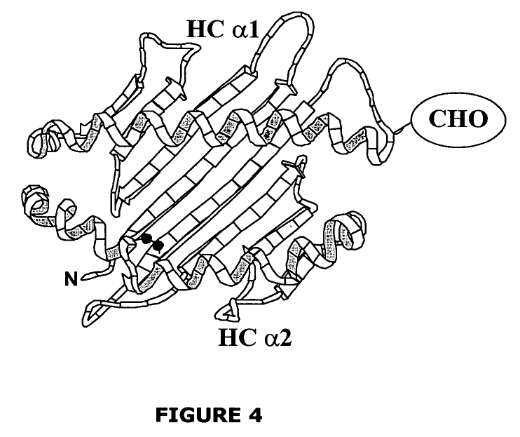

HLA/MHC genes are the most polymorphic system in mammals, generated by systematic recombinatorial and point mutation events; as such, hundreds of different HLA types exist throughout the world's population, resulting in a large immunological diversity. Individuals inherit a set of three class I genes from each parent, and since their expression is codominant, a single person may therefore display up to six different HLA class I molecules upon his or her nucleated cells. Such extensive HLA diversity results in differing susceptibilities and/or resistances between individuals in infectious diseases. Depending upon allelic composition, two individuals' molecules may not necessarily bind the same peptides with equal affinity or even at all. Therefore, despite the overall structural conservation illustrated among class I heavy chains, their peptide binding grooves can vary drastically from one allelic form to another; as a result various isoforms are capable of associating with distinct arrays of peptides. A binding platform is shown in FIG. 4. The first two domains (alpha 1, alpha 2) of the heavy chain create the peptide binding cleft and the surface that contacts the T-cell receptor. X-ray crystallographic analysis indicates that a processed antigen is presented as a peptide bound in a cleft between the two α-helices of the heavy chain of the HLA complex (Bjorkman P. J., 1987; Nature 329: 506-512 & 512-518/Garett T. J. 1989: Nature 342; 692-696/Saper M. A.; 1991; J. Mol. Biol. 219; 277-319/Madden D. R. (1991) Nature 353; 321-325; the contents of each are herein expressly incorporated by reference in their entirety.). The third domain (alpha 3) associates with the T-cell co-receptor, CD8, during T-cell recognition. Availability of a wide spectrum of recombinant sHLA molecules overcomes the current art limitations on population coverage imposed by the rules of MHC restriction. In most cases, a single MHC molecule will be useful only for treating/testing a small subset of patients who express antibodies capable of binding that specific MHC molecule. Since every individual has differing MHC molecules, will have been exposed to different MHC molecules, and will have antibodies to different MHC molecules, the screening of numerous individual MHC molecules is a prerequisite for understanding the difference in disease susceptibility between individuals.

### Purification Methodology

There are many purification methods available for the separation of macromolecules. To effectively resolve a crude mixture of substances, it may be necessary to use a combination of techniques. In most cases, a purification procedure will involve some chromatographic techniques.

Affinity chromatography occupies a unique place in separation technology since it is the only technique which enables purification of almost any biomolecule on the basis of its biological function or individual chemical structure. Affinity chromatography makes use of specific binding interactions that occur between molecules. It is a type of adsorption chromatography in which the molecule to be purified is specifically and reversibly adsorbed by a complementary binding substance (ligand) immobilized on an insoluble support (matrix). A single pass through an affinity column can achieve a 1,000-10,000 fold purification of ligand from a crude mixture. It is possible to isolate a compound in a form pure enough to obtain a single band upon SDS-polyacrylamide gel electrophoresis. Any component that has an interacting counterpart can be attached to a support and used for affinity purification.

Successful separation by affinity chromatography requires that a biospecific ligand is available and that it can be covalently attached to a chromatographic bed material called a matrix. It is important that the biospecific ligand (antibody, enzyme, or receptor protein) retains its specific binding affinity for the substance of interest (antigen, substrate, or hormone). Methods must also include removing the bound material in active form with low pH, high pH, or high salt. The selection of the ligand for affinity chromatography is influenced by two factors. Firstly, the ligand should exhibit specific and reversible binding affinity for the substance to be purified. Secondly, it should have chemically modifiable groups, which allow it to be attached to the matrix without destroying its binding activity. The ligand should ideally have an affinity for the binding substance in the range 10⁻⁴ to 10⁻⁸ M in free solution.

The protocol herein discussed provides a method to couple protein to a commercially available CNBr-activated SEPHAROSE® 4B (APB #17-0430-01). An alternative option would be running the procedure with SEPHAROSE® 4 Fast flow (APB #17-0981-01). SEPHAROSE® Fast Flow is more highly crosslinked than SEPHAROSE® 4B. As a result, Fast Flow beads are more stable and can withstand higher flow rates than the 4B beads. CNBr-activated SEPHAROSE® 4B is better suited for batch chromatography and small columns with gravity flow. Another difference is in coupling capacities. The coupling reaction proceeds most efficiently in the pH range 8-10 where the amino groups on the ligand are predominantly in the unprotonated form. A buffer at pH 8.3 is most frequently used for coupling proteins. IgGs are often coupled at a slightly higher pH, for example in a NaHCO₃ buffer (0.2-0.25 M) containing 0.5 M NaCl, at pH 8.5-9.0. Carbonate/bicarbonate and borate buffer systems with the addition of NaCl may be used. The coupling buffer solution should have a high salt content (about 0.5 M NaCl) to minimize protein-protein adsorption caused by the polyelectrolyte nature of proteins. Coupling at low pH is less efficient but may be advantageous if the ligand loses biological activity when it is fixed too firmly, e.g. by multi-point attachment, or because of steric hindrance between binding sites which occurs when a large amount of high molecular weight ligand is immobilized. A buffer of approximately pH 6 is used. Tris and other buffers containing amino groups must not be used at this stage since these buffers will couple to the gel.

Protein coupled to CNBr-activated SEPHAROSE® 4B is usually more stable to denaturation than the protein in free solution, but reasonable care in the choice of storage conditions should be exercised. Suspensions should be stored in a refrigerator below 4° C. in the presence of a suitable bacteriostatic agent. The choice of buffer solution depends on the properties of the particular coupled protein.

In affinity chromatography, nonspecific proteins flow through the column while the specific protein is retained by the column. The protein is then eluted, and individual fractions are tested for specific-binding activity and purity. Several different approaches can be taken to allow efficient binding of antigens to immunoaffinity columns. Because the antibody is not in solution, the time required for the antibody-matrix/antigen interaction will have different kinetics than soluble interactions. It will take considerably longer for equilibrium to be reached than for solution assays. Therefore, the binding protocol should maximize the degree of interaction. The recommended method is binding by passing the antigen solution down an antibody-matrix column, keeping the antigen in contact with the antibody for as long as possible. In this case, high-affinity antibodies will be significantly more efficient at removing the antigen from solution than low-affinity antibodies. Several small-scale columns can be used to determine the best conditions for binding and collecting the antigen.

Although the exact affinity of an antibody for an antigen can be calculated, for most work the crucial criterion is whether the antibodies will remove the antigen from solution quantitatively. The easiest method to test this is to set up small-scale reactions and examine the first wash buffer for the presence of the antigen. The amount of bound antigen may be increased by using higher amounts of antibodies on the beads, by increasing the number of beads, or by increasing the amount of time for binding. Unfortunately, all of these conditions will raise the nonspecific background, so a compromise normally will result in the highest yields with the lowest acceptable background. Use of high-affinity antibodies solves the problem of efficiently collecting the antigen. Consequently, they can be used in dilute solutions, at relatively lower concentrations, and for shorter times.

A titration can be performed as a first step in estimating the ratio of column matrix needed to bind a given amount of antigen. This can be handled where an equal volume of the antibody/SEPHAROSE® 4B matrix is added to samples containing increasing concentrations of the antigen. The slurry is mixed at 4° C. for 1 hr and then processed. This will yield a rough idea of the volume of column matrix needed to collect the desired amount of antigen. If the supernatants from the binding reaction are assayed for the presence of the antigen, the extent of antigen depletion also can be determined.

Developing the best elution conditions is an empirical task determined by testing a series of buffers. Three types of elution are possible. The antigen-antibody interactions can be broken by (1) treating with harsh conditions, (2) adding a saturating amount of a small compound that mimics the binding site, and/or (3) treating with an agent that induces an allosteric change that releases the antigen. The most commonly used elution procedure relies on breaking the bonds between the antibody and antigen by pH.

The mildest elution conditions are required if the protein of interest is labile. Avoid dithiothreitol and other reducing agents, as they will break disulfide linkages if present within the molecule of interest. Any buffers that fail to elute the antigen should be considered as good candidates for wash buffers. Some noneluting buffers may, in fact, drive the antibody-antigen equilibrium toward complex formation. The usual procedure when elution conditions have not been defined is to try the mildest elution conditions first and proceed to harsher treatments. If trying for the gentlest elution conditions, start with acid conditions first, then check basic elution buffers. If these conditions do not elute the antigen, try others. A general order to check the various conditions would be:

| Low pH | acid, pH 3-1.5 | |
|---|---|---|
| | 0.1 M glycine-HCl (pH 2.5) | |
| | 0.1 M glycine sulfate (pH 2.3) | |
| | 0.1 M propionic acid (pH 2.3) | |
| | 3.0 M KSCN (pH 2.3) | |
| High pH | base, pH 10-12.5 | |
| | 0.1 M glycine-NaOH (pH 11.0) | |
| | 0.15 M NH₄OH (pH 10.5) | |
| Chaotropic | MgCl₂, 3-5 M | 4 M MgCl₂ in 10 mM PBS (pH 7.0) |

| | |
|---|---|
| Agents | LiCl 5-10 M |
| | Water |
| Polarity- | Ethylene glycol 25-50% |
| reducing Agents | Dioxane 5-20% |
| Denaturing | Thiocyanate 1-5 M |
| Agents | Guanidine 2-5 M |
| | Urea 2-8 M |
| | SDS 0.5-2% |

Microconcentrators are used primarily for removal of excess salts in protein purification or analysis. A variety of materials have been used to fabricate these semipermeable membranes, ranging from cellulose and cellulose esters to polyethersulfone (PES) or polyvinylidene difluoride (PVDF). All membranes are characterized by their molecular-weight cutoff (MWCO) value. This is usually defined as the molecular weight of a solute that is 90% prevented from penetrating the membrane under a chosen set of conditions. How readily a particular protein is rejected by the membrane is a function of the shape, hydration state, and charge of the protein molecule. Moreover, MWCO values are not sharp; rather, there is a gradual increase in retention as the size of solute molecules approaches and exceeds the average membrane pore size. Only at the point where all pores are smaller than a particular solute molecule is that molecule completely excluded.

The advantage of desalting processes based on ultrafiltration over those based on simple dialysis is that the rate of low-molecular-weight solute removal is not determined by a concentration differential, but rather by the flow rate of solvent and the rejection of the solute by the ultrafiltration membrane employed. Membranes for ultrafiltration are generally selected on the basis of the MWCO needed to retain the protein of interest but allow the maximum amount of other materials to pass through. It is usually best to choose an MWCO value that is roughly one-half the molecular weight of the species to be retained. This provides a reasonable margin of retention whereby almost none of the protein of interest should be lost, but at the same time provides the largest difference between the MWCO value and the molecular weight of the salts to be removed, thereby maximizing filtration rate.

In regard to the degree of nonspecific adsorption of protein to membranes, losses of 1% to 5% are not uncommon when dealing with total quantities of protein in the range of 1 to 10 mg using a filter with a 43-mm diameter. The nature of the buffer can also affect adsorption of protein; some membranes exhibit altered flow properties when high levels of ions are present. In this regard, phosphate buffers seem to present more of a problem than Tris buffers. The degree of concentration to be achieved by ultrafiltration should be that required for subsequent work. Recovery of sample following concentration is generally 95%; failure to achieve this value usually indicates leakage into the filtrate or nonspecific binding to the membrane and/or concentration apparatus itself.

At a constant temperature and pressure, the flow rate is a function of the filter area and the degree to which concentration polarization can be avoided. Buildup of protein on the surface will result in slow filtration, even when the protein concentration of the sample is relatively low. Filtration rates at 4° C. are often only one-half those seen at 25° C. because of the influence of viscosity.

For biochemical analysis, monoclonal antibodies are particularly useful for identification of HLA locus products and their subtypes. W6/32 is one of the most common monoclonal antibodies (mAb) used to characterize human class I major histocompatibility complex (MHC) molecules (ATCC® No. HB-95™, American Type Culture Collection, Manassas, Va.). This antibody recognizes only mature complexed class I molecules. It is directed against a conformational epitope on the intact MHC molecule that includes both residue 3 of β₂m and residue 121 of the heavy chain (Ladasky J J, Shum B P, Canavez F, Seuanez H N, Parham P. Residue 3 of beta2-microglobulin affects binding of class I MHC molecules by the W6/32 antibody. Immunogenetics 1999 April; 49(4):312-20, the contents of which is expressly incorporated herein by reference in its entirety.). Some HLA-C molecules could not be clearly identified in immunoprecipitations with W6/32, suggesting that these HLA-C locus products may be associated only weakly with β₂m, explaining some of the difficulties encountered in biochemical studies of HLA-C antigens. The polypeptides correlating with the C-locus products are recognized far better by HC-10 than by W6/32 which seems to confirm that at least some of the C products may be associated with β₂m more weakly than HLA-A and -B.

HC-10 is reactive with almost all HLA-B locus free heavy chains. The HLA-A heavy chains are not as strongly recognized by HC-10 as B alleles, but seem to react well with free heavy chains of HLA-C types. No evidence for reactivity of HC-10 with heavy-chain/β2m complex was obtained. None of the immunoprecipitates obtained with HC-10 contained β2m. This suggests that HC-10 is directed against a site of the HLA class I heavy chain that might include the portion involved in interaction with β2m. The pattern of HC-10 precipitated material is qualitatively different from that isolated with W6/32.

TP25.99 detects a determinant in the alpha3 domain of HLA-ABC. It is found on denatured HLA-B (in Western) as well as partially or fully folded HLA-A, B, & C. It doesn't require a peptide or β₂m, i.e., it works with the alpha 3 domain which folds without peptide. This makes it useful for HC determination.

Anti-human β2m (HRP) (DAKO P0174) recognizes denatured as well as complexed β₂m. Although in principle anti-β2m reagents could be used for the purpose of identification of HLA molecules, they are less suitable when association of heavy chain and β2m is weak. The patterns of class I molecules precipitated with W6/32 and anti-β2m are usually indistinguishable.

### EXPERIMENTAL EXAMPLES

### Purification of Individual, Soluble MHC Molecules

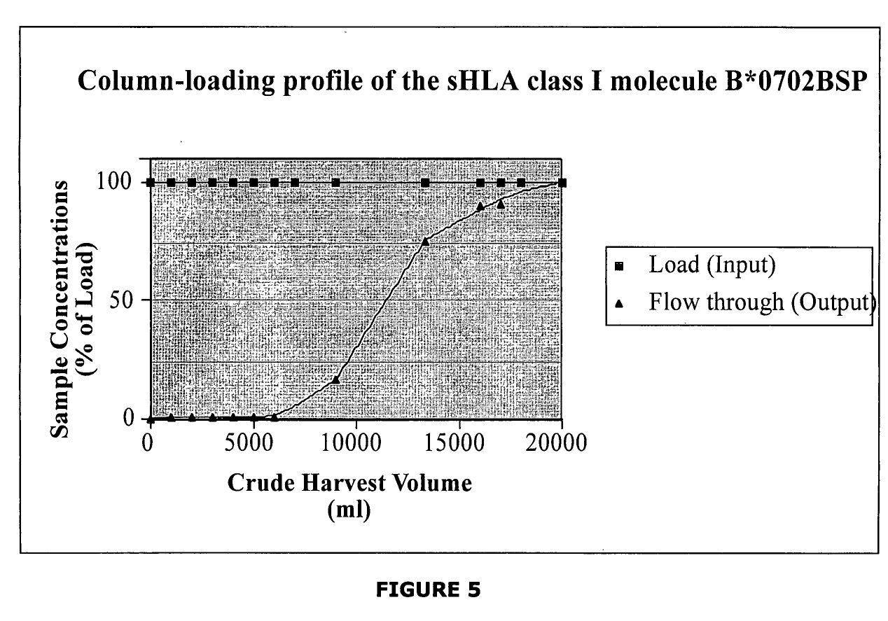

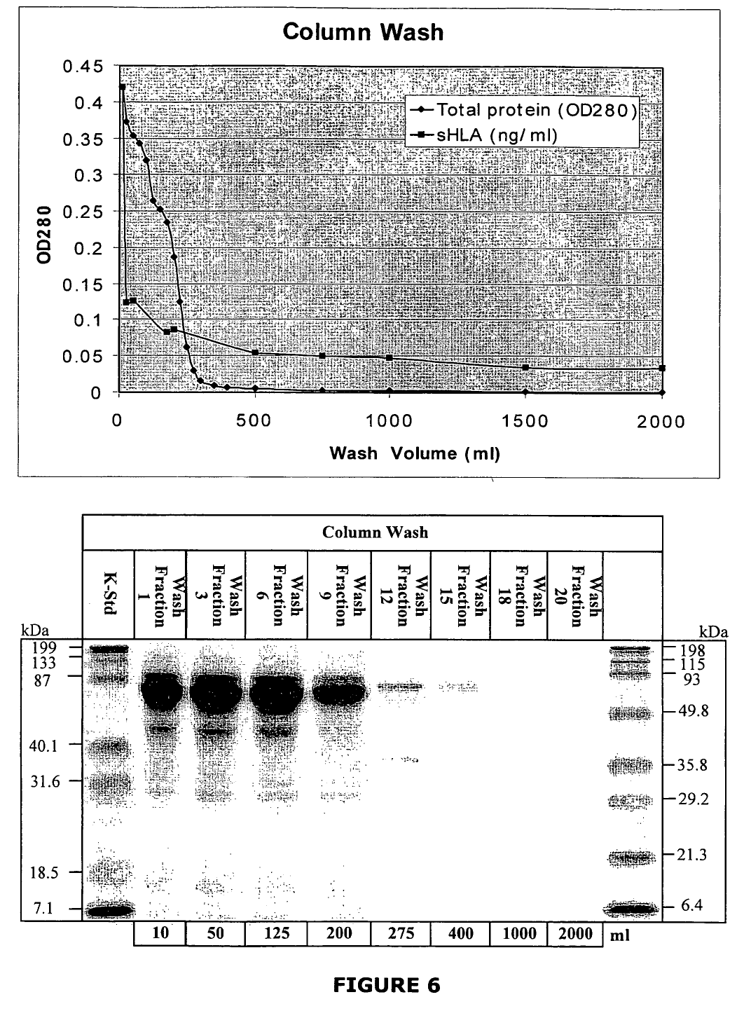

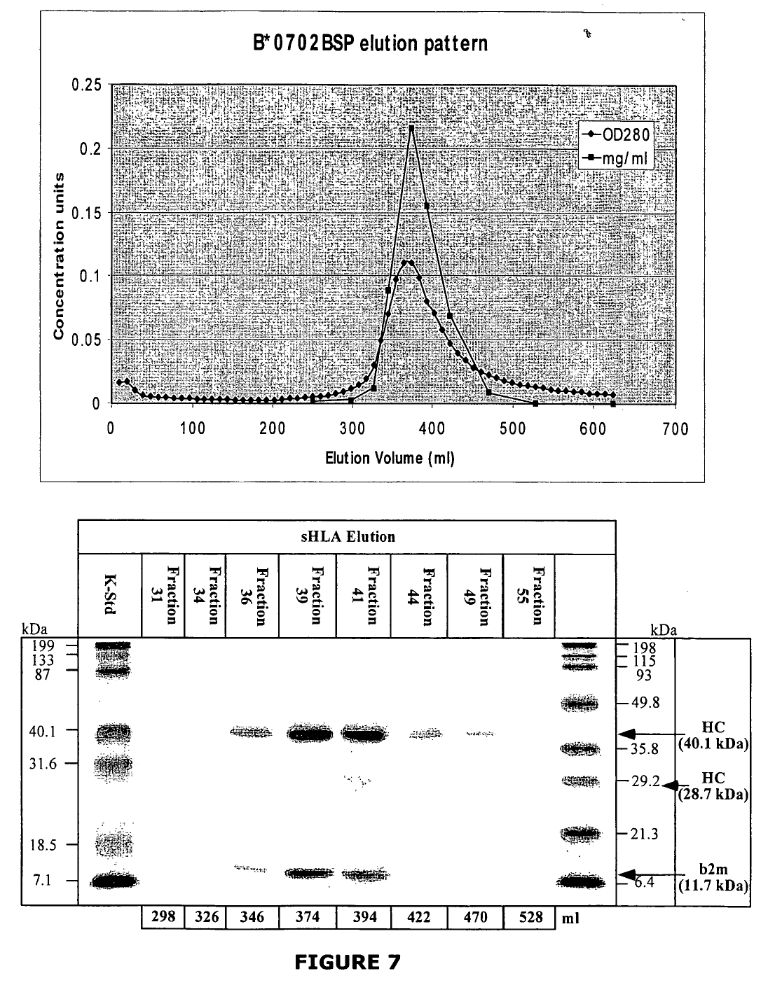

The present invention is directed to a unique method for producing, isolating, and purifying class I molecules in substantial quantities. As an example of the method of the present invention, the following graphs show that the test allele B*0702BSP produced in static culture can be purified to homogeneity and eluted as intact molecule. FIG. 5 demonstrates that a W6/32-coupled affinity column can be saturated with crude harvest containing sHLA. Individual values were determined through a standardized sandwich ELISA procedure using W6/32 as capturing antibody and anti-β2m as detecting antibody. This ELISA procedure allows only the detection of intact sHLA molecules. After successful loading, the column is washed with PBS. FIG. 6 shows the washing step. The removal of total protein and active sHLA measured through OD₂₈₀ and ELISA, respectively, can be followed. It shows that after 500 ml of wash volume, impurities are successfully removed from the column. This was also confirmed through SDS-PAGE analysis of the wash fractions collected. In FIG. 7, we were able to elute sHLA molecules with 0.1 M glycine (pH 11.0) and neutralize in 1 M potassium phosphate (pH 7.0) that resulted in fractions of intact molecules as shown through the standard ELISA procedure as well as OD₂₈₀ detection. Elution occurred in a single peak indicating the absence of nonspecifically bound material on the column. SDS-PAGE analysis confirmed the size of the subunits and their purity. The final MACROCEP® procedure was used to remove the neutralization buffer and replace it with PBS (0.02% Sodium azide). This buffer is highly suitable to maintain structural integrity and maintain the stability of the sHLA complex.

The same procedure is used to finally concentrate the protein to increase its stability. Higher concentrations are usually more suitable in most applications. All MACROCEP®'s wash flow-through's have minimal sHLA content and are usually discarded after the procedure. To remove possible particles or bacterial growth, filtration through a 0.2 micron filter is standard procedure. With this purification run, an overall efficiency of 75% was achieved.

### Chemical and Physical Purity of Individual, Soluble MHC Molecules

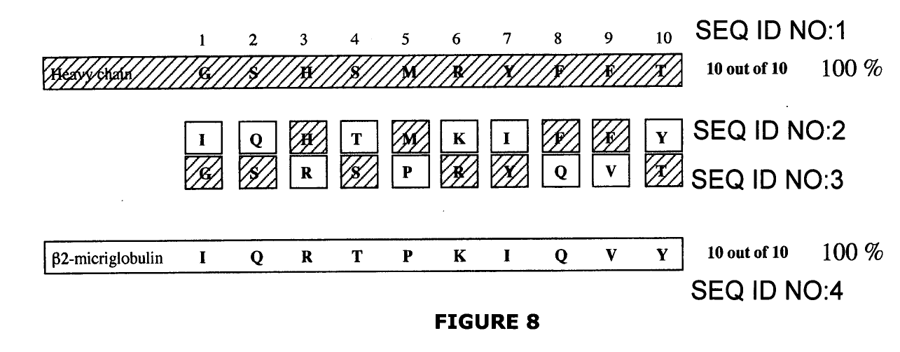

To confirm that the sHLA produced and purified by the method of the present invention are correctly translated, an Edman degradation was performed to receive the sequence of the first 10 amino acids. Since an intact sHLA molecule is a complex consisting of HC, β2m and a peptide, sequencing results gave us several different amino acids at each position. Since HC and β2m are present in a ratio of 1:1 each position from 1 to 10 should predominantly contain both HC and β2m amino acids in about equal amounts. Since both sequences are published and well known, a comparative analysis can easily be done. Because sHLA molecules bind a variety of different peptides, these amino acids are producing noise at each position rather than delivering distinctive recognizable amino acids. FIG. 8 illustrates protein sequence data for MHC Class I HLA-A*0201T. The comparison clearly shows that this sHLA molecule is correctly translated at the amino terminal end.

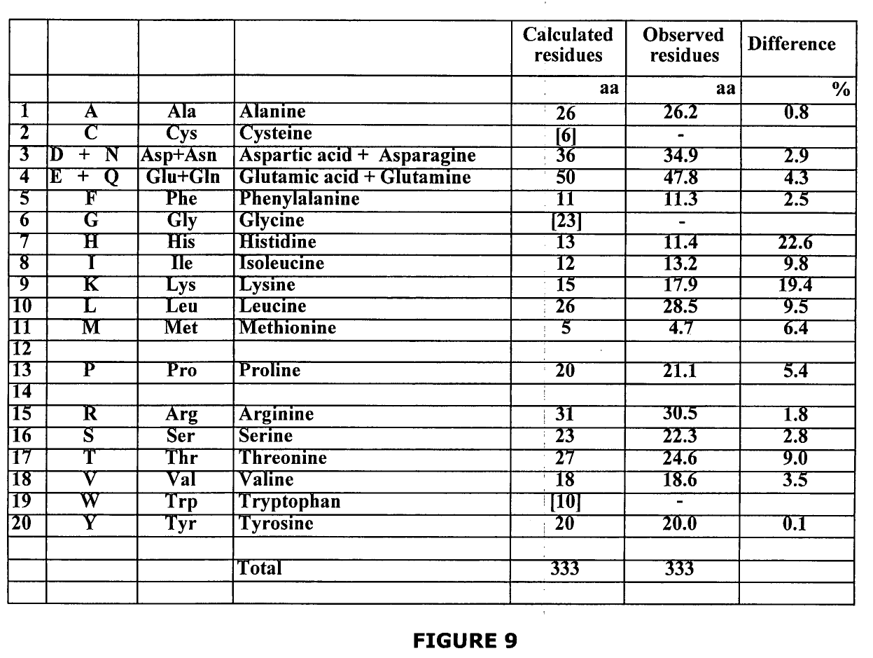

Proteolysis of the whole molecule complex and analysis of the amino acid composition was executed on B*1512T (FIG. 9). The procedure showed a close relationship between the amino acid content of the calculated versus the observed residues suggesting a full length molecule. During the procedure, some amino acids were expectedly degraded and were not taken into consideration. The close match is a good indication of the purity of our test-sample and evidence that no other major impurities were present in the sample.

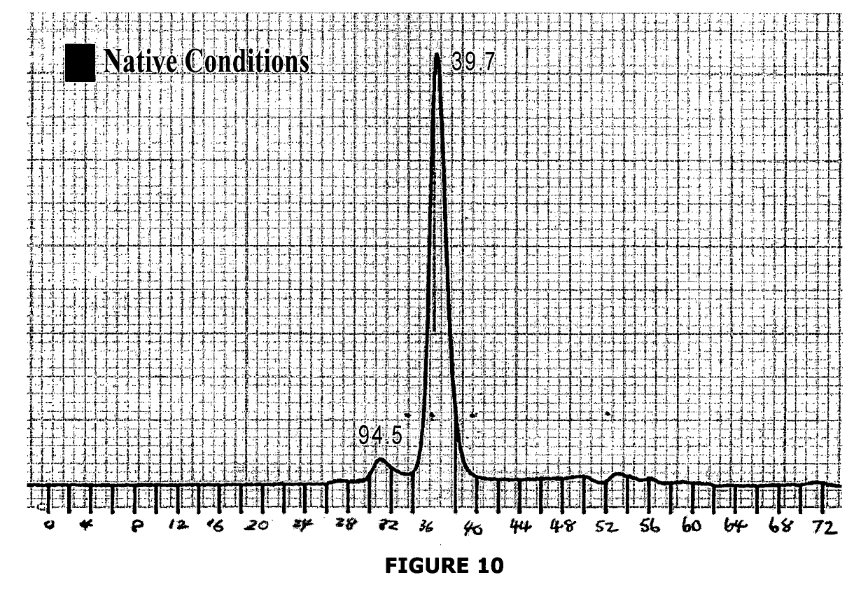

The sHLA's produced and purified by the method of the present invention were analyzed by SUPERDEX® chromatography to demonstrate sample purity (FIG. 10). The SUPERDEX®-FPLC analysis of B*1512T under native conditions showed a characteristic peak corresponding to the sHLA complex. No other major bands can be detected confirming the pure nature of our preparation. Under such native conditions, a peak of the size of 39.7 kDa is seen, which is in the area of complexed sHLA. No bands at 31 kDa, representing free HC, or at 12 kDa for β2m are visible. However, a minor band at approximately 94.5 kDa can be seen, which represent aggregated HCs. Because sHLA samples are filtered through a 10 kDa filter during the MACROCEP® procedure, these free HC molecules remain in the solution and cannot be removed. Aggregated HC's are not considered an impurity of the sample. In addition, their contribution to the final protein amount is less than 1%. The overall purity of the complex compared to foreign proteins is more than 99.9%.

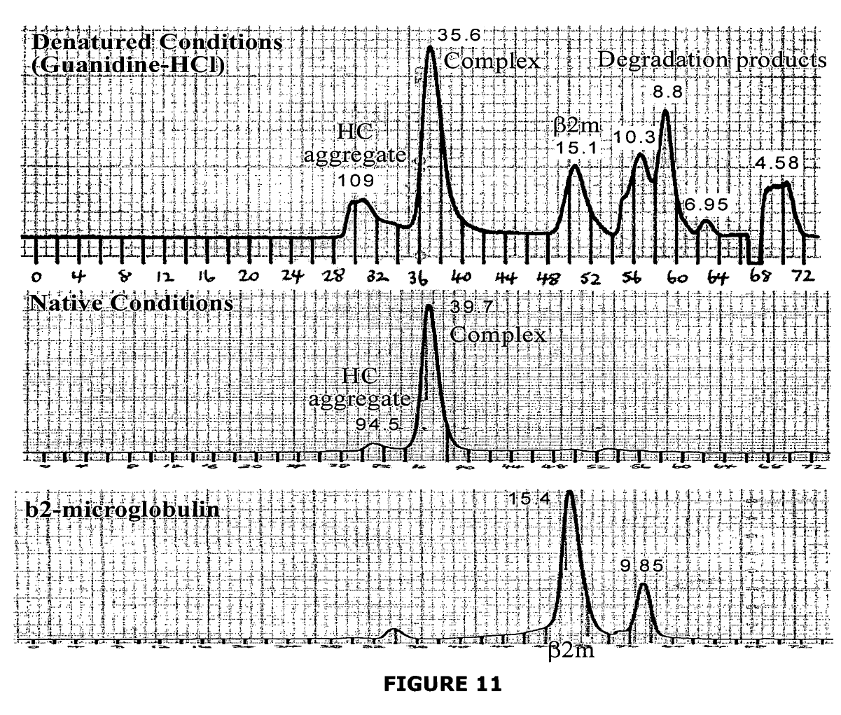

A triple analysis of B*1512T is presented in FIG. 11. It shows a separation of sHLA under denaturating and under native condition as well as separation of purified free β₂m (Serotec) alone. A standard curve was run in parallel to estimate molecular weights (not shown).

Using guanidine-HCl as additive to denature the probe, the sample of B*1512T was run under equal conditions as the other samples. The results seen demonstrate that the sHLA complex is unexpectedly stable under such denaturing conditions. A clear peak resembling the pure complex can be identified which is at the same position as the native peak. As expected, some sHLA complexes do fall apart, which resulted in the increase of aggregated HC and an increase in free β2m as their positions are identified through their overlap with the native samples. Again, the denaturation process did not deliver a peak at 31 kDa corresponding to free HC, suggesting that HC monomers aggregate to a higher size complex. During the denaturing process, several peaks of lower molecular weight appeared, which probably correspond to aggregated peptides released from the destroyed molecules and/or through fragmentation of β2m and HC subunits.

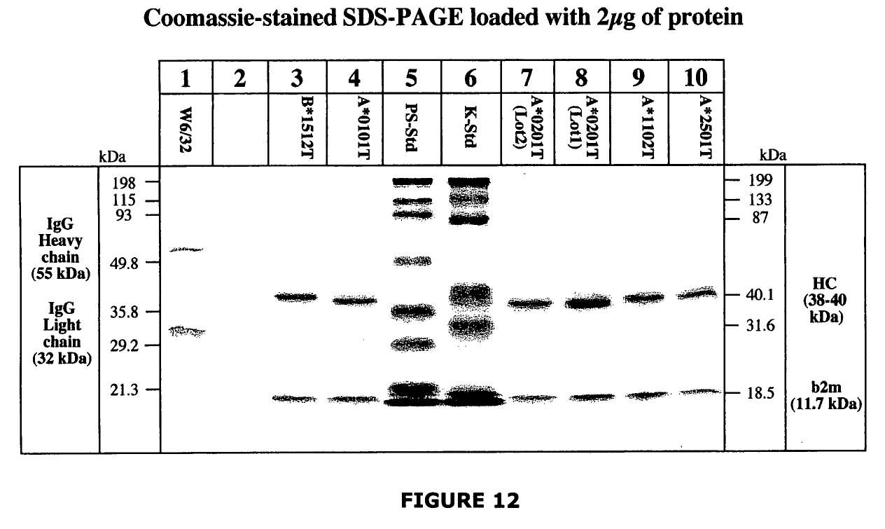

Several sHLA alleles were loaded on an SDS-PAGE gel and stained with Coomassie to assess sample purity (FIG. 12). A band for HC and β₂m, respectively, was detected demonstrating the purity of all samples tested. The antibody W6/32, which is used in the process of affinity purification, is also added. In none of the samples could an equal band be detected, thus showing that leakage of W6/32 during elution does not occur.

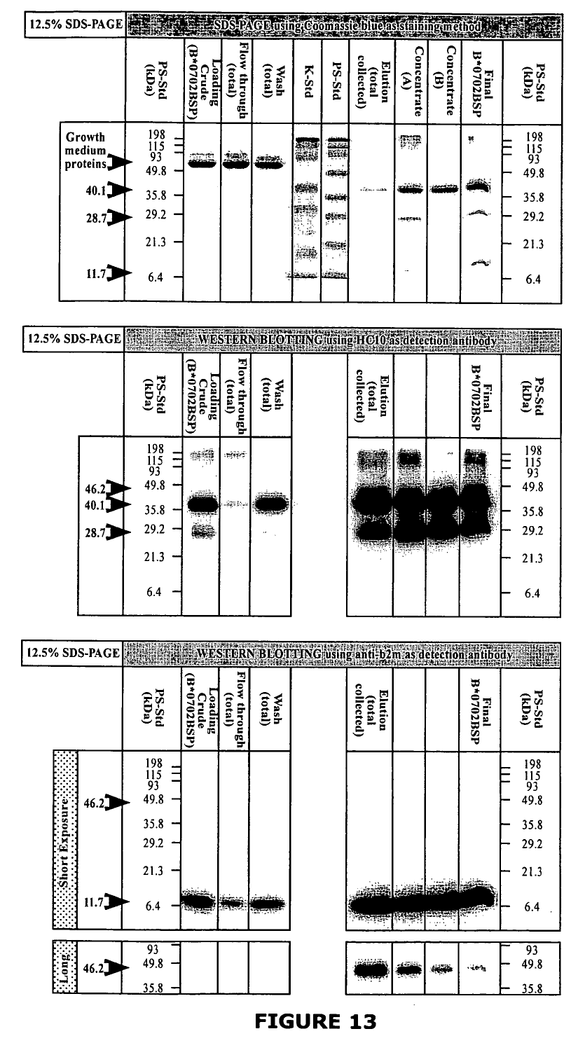

Western blot analysis to follow the HC and β2m subunits of sHLA were also performed (FIG. 13). The upper portion shows the results of an SDS-electrophoresis performed running crude harvest (load), the flow through (output of the column) and the wash on the left side, elute, concentrate and final sample on the right.

Using HC10 antibody visualized with a secondary mouse antibody coupled to HRP, several bands could be stained resembling different aggregates of HC. It appears that the dimeric form is dominant (40.1 kDa) over the monomeric form (28.7 kDa) after denaturation and SDS treatment. The lower value for the dimeric form is evidently an artifact and caused by an aberrant running behavior on SDS-PAGE gels since a consistent amount of SDS is not any more bound per unit weight of protein. The carbohydrate moiety attached to the HC might also be involved. Higher aggregates are also visual to a minor extent. The results show that sHLA is present in the crude and binds to the column since there is a drastic reduction in signal observed in the flow through. Saturation of the column does result in material leaving the column not captured. Therefore, wash fractions will also contain some sHLA not captured. The protein is highly concentrated in the purified sample and concentrates do not look different than eluted molecules.

An anti-β2m antibody directly labeled with HRP was used to visualize the lighter subunit. A single band of 11.7 kDa was seen as expected. β2m does not seem to aggregate. However, a faint band at 46.2 kDa could be observed. An extended exposure showed a clear band at this location which is in the size of the intact complex. This would suggest that some complexes survived the denaturation step and show SDS resistance.

Separation under denaturing conditions and staining with the antibodies HC10 and anti-β2m revealed that both the heavy chain and β₂m are present. The secondary antibody directed against mouse antibodies also did not reveal any additional bands, indicating that the preparation is free of possible W6/32 antibody contamination, which was used in the purification step

### Functional Purity of Individual, Soluble MHC Molecules

It is important to demonstrate that the individual, soluble MHC molecules produced, isolated and purified according to the methods of the present invention function in various assays. Functional purity of the individual, soluble MHC molecules produced as described herein above is demonstrated by three methods: (1) chaperone interaction experiments demonstrating that truncating the HLA molecule does not alter the quaternary structure of the class I protein; (2) Edman and Mass Spec amino acid sequencing of the peptides eluted from the sHLA class I molecules, demonstrating that the peptide motifs match those previous shown for membrane bound class I molecules; and (3) peptide binding assays demonstrating that sHLA will exchange endogenous peptide ligands for synthetic peptide epitopes of known high affinity. The results from these three sets of experiments demonstrate that (1) sHLA function in other assays in the same manner as do cell surface HLA; and (2) that synthetic peptides bind specifically to their cognate class I sHLA molecules, thereby demonstrating that the sHLA molecules produced and purified by the methods of the present invention bind peptide in the manner specific for each HLA molecule.

1. Chaperone interaction experiments

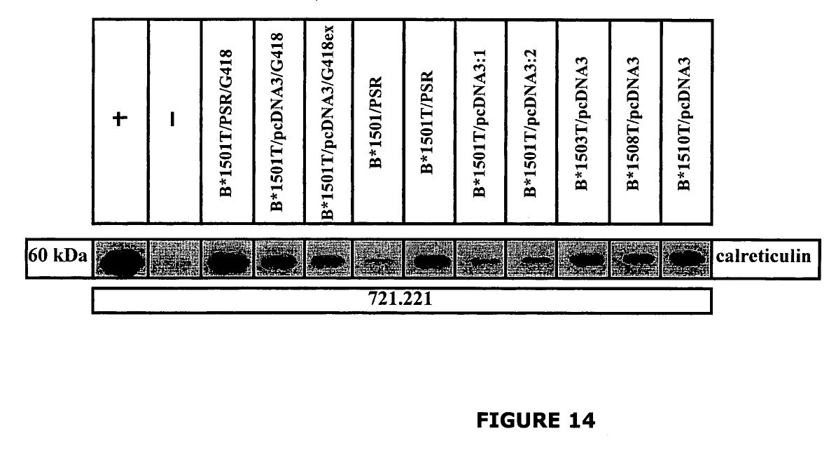

The class I molecule interacts with several chaperones as it traffics through the cell on its way to the cell surface. These chaperones include, but are not limited to, calnexin, calreticulin, Tapasin, and Erp 94. ³⁵S pulse chase/immunoprecipitation experiments were performed to demonstrate that the sHLA class I proteins produced and purified by the method of the present invention interact with chaperones normally. Interaction with calreticulin, calnexin, and tapasin has been demonstrated, and interaction with calreticulin is shown in FIG. 14.

### 2. Edman and Mass Spec Amino Acid Sequencing

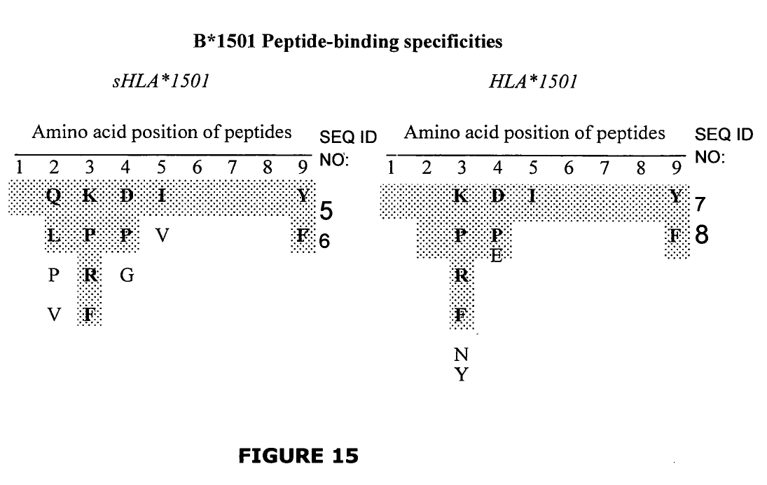

The peptides bound in the antigen binding groove of the class I molecule impact the conformation and the antibody reactivity of the class I molecule. The peptides eluted from the sHLA class I molecules produced and purified by the methods of the present invention have been characterized, and it was found that the peptide motifs match those of membrane bound class I molecules reported by other laboratories. FIG. 15 shows a motif comparison between sHLA-B*1501 purified by the methods of the present invention and a membrane bound B*1501 motif from another laboratory. The motifs are nearly identical. The same result has been seen with six sHLA class I molecules analyzed. In addition, individual peptide ligands isolated from the sHLA purified by the methods of the present invention have been sequenced, and they match ligands found in membrane bound class I molecules of other laboratories. Thus, the sHLA proteins of the present invention appear to traffic and bind peptides as do membrane bound class I.

### 3. Peptide Binding Assays

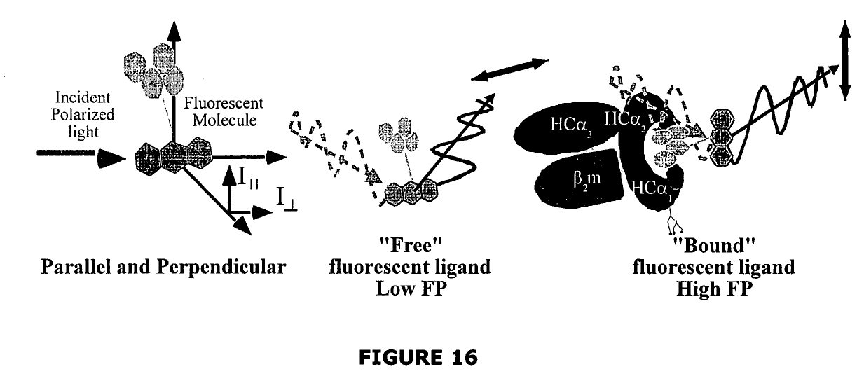

Fluorescence polarization allows the direct measurement of the ratio between free and bound labeled ligand in solution without any separation steps (FIG. 16). Most important, FP allows real time measurements of single reactions to determine binding kinetics as well as equilibriums. Such constants can be used to directly establish the quality of sHLA molecules and also allow the comparison to native HLA molecules.

The technique of FP is based on the fact that if excited with plane-polarized light, the light emitted by a fluorophore is polarized as well. FP values are defined by the equation:

$$Polarization = \frac{{I}_{∥} - {I}_{⊥}}{{I}_{∥} + {I}_{⊥}}$$

where I_{∥} is the intensity of the fluorescence measured in the parallel (∥) or horizontal direction (S) and I_{⊥} is the intensity of the fluorescence measured in the perpendicular (_{⊥}) or vertical direction (P).

If a fluorescent-labeled peptide binds to the sHLA molecule of higher molecular weight, the average angle (composed of the distribution of all angles between the optical planes) will decrease due to the slower molecular rotation of the bound probe (FIG. 16). Therefore, the ratio between the bound and free probe can be measured by FP directly in solution. This advantage makes FP an excellent tool for the fast and precise determination of molecular interactions between sHLA and peptide.

A binding assay was developed to demonstrate that the labeled probe will bind to the molecule of interest. Positive binding events of synthetic peptides to sHLA are a clear confirmation of the molecular specificity of the sHLA molecules tested. In addition, binding of defined peptides also demonstrates structural integrity of the trimeric complex. Such quality assurances are of key importance in utilizing sHLA molecules for sera screening applications.

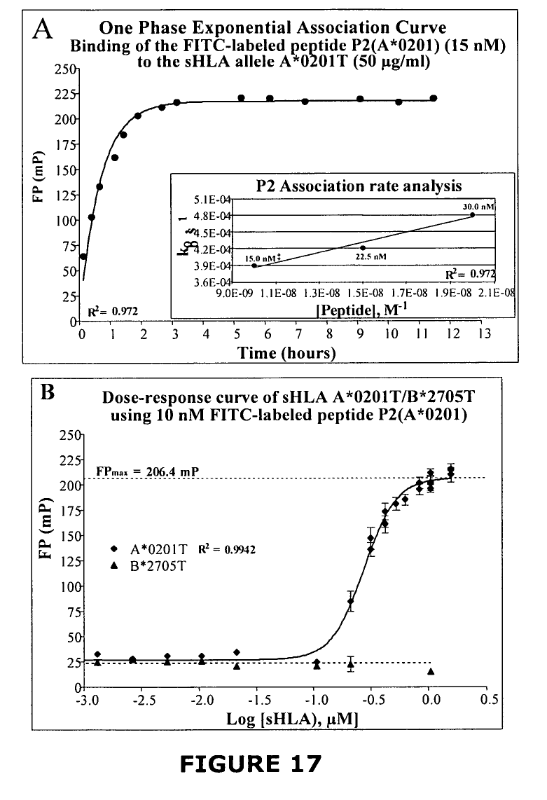

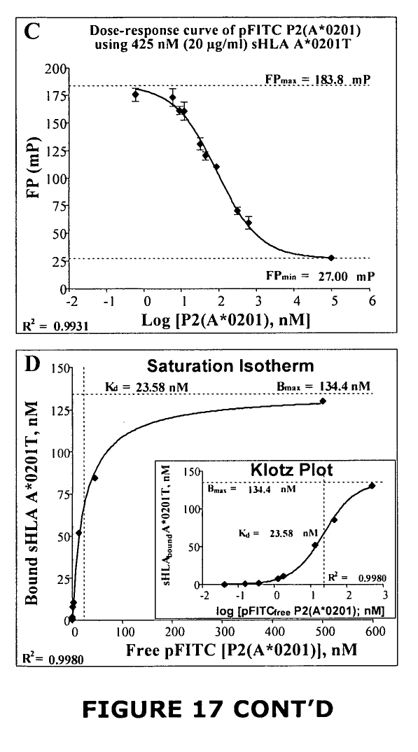

As a first quality control, FP is a suitable method to determine kinetics using real time analysis. Kinetic experiments provide a more sensitive test than equilibrium experiments and give information on the rate constants of the interaction. Kinetic experiments were performed herein that determined the binding of the specific pFITC P2(A*0201) peptide to sHLA A*0201T as a function of time to prove that the sHLA molecules of the present invention are highly functional (FIG. 17A) and capable of forming a trimeric complex. To obtain the observed (apparent) association rate constant (k_{ob}) value, constant amounts of the peptide ligand and sHLA (50 μg/ml) were incubated together, and binding was monitored over time. Binding parameters were determined by fitting all data points to a mono-exponential association model (Y=Yₘₐₓ(1−e^{-kt})). Under the chosen conditions, the association rate constant was 0.914 10⁴ [M⁻¹s⁻¹], and the dissociation rate constant was 2.94 10⁻⁴ [s⁻¹], resulting in an equilibrium dissociation constant of 32.2 nM.

To determine the sHLA concentration necessary to yield maximal peptide binding conditions, fixed amount of fluorescent-labeled peptide was incubated with varying concentrations of sHLA (FIG. 17B). Results show that the sHLA concentration can be saturated, further confirming its functionality. Furthermore, the sHLA allele A*0201T was exchanged with B*2705T, which was reported to bind a different ligand repertoire. While the addition of more sHLA resulted in a gradual increase in fluorescence polarization, same amounts of the non-specific allele B*2705T did not have any effect on the polarization of the pFITC conjugates, indicating that the enhanced polarization was a result of specific binding.

The effect of varying the fluorescent-labeled peptide on the level of binding observed was also tested. The saturation binding curve for the fluorescent-labeled peptide P2(A*0201) where FP readings were plotted as a function of pFITC concentrations at a fixed concentration of 425 nM (20 μg/ml)ₛHLA A*0201T is shown in the FIG. 17C. Since FP measurements directly detect the ratio between bound and free fluorescent-labeled ligand, the FP signal is greater for low ligand concentrations. Accordingly, binding of pFITC conjugates to sHLA was characterized by an initial upper plateau for the bound state with highest polarization values followed by a steady polarization decrease as a result of the presence of increasing amounts of free fluorescent-labeled peptide ligand. To obtain the K_{d} for sHLA/pFITC interactions, we used the recently described FP K_{d} model from Prystay et al. 2001 (FIG. 17D). A K_{d} of 23.6 nM was established. Matching kinetic and equilibrium studies show that sHLA molecules are a real alternative to native molecules for a sera screen platform.

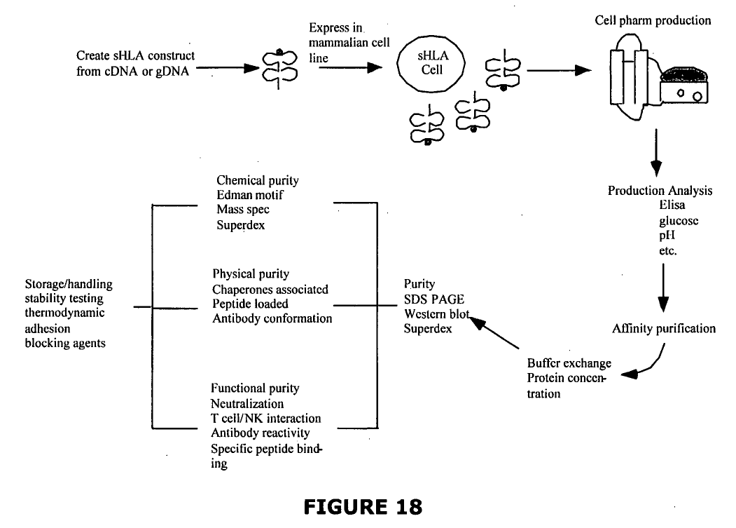

In summary, shown in FIG. 18 is a general outline of the purification and characterization procedures of soluble human HLA proteins of the present invention. The first steps involve sHLA construct design and transfection procedures followed by large scale production of sHLA molecules in cell pharms. The sHLA is then purified by affinity column purification (which includes the steps of loading, washing and elution) and buffer exchange and concentration of purified allele using MACROCEP® concentration filters. The pure protein is then sterile filtered, aliquoted and stored, and the concentration of the stored pure protein is determined. Finally, quality control demonstrating the extent of chemical purification is performed using techniques known to those of ordinary skill in the art, including but not limited to, SDS-PAGE, Western blot analysis, SUPERDEX® chromatography to demonstrate sample purity, and the like.

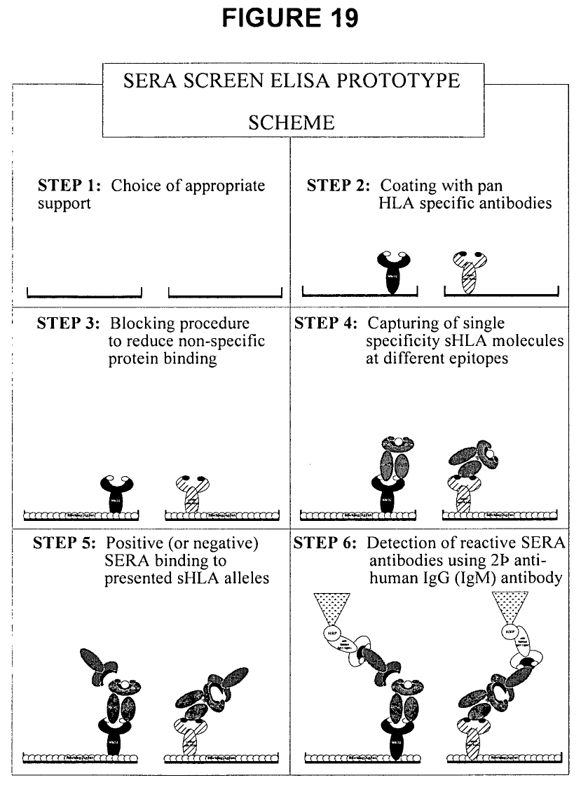

### DETAILED DESCRIPTION OF FIGS. 19-29

### Anti-HLA Assay Using Individual Soluble HLA Molecules

Sandwich assays can be used to study a number of aspects.

Antibodies available to different epitopes or subunits of a heteropolymer can be used to present a complexed molecule in different ways. Such sandwich assays can be designed to test for the presence of sera antibodies recognizing the molecules captured with a first antibody. A graphical representation of a sandwich ELISA assay in which sHLA molecules are bound to a plate for detection of anti-HLA antibodies in a sample is shown in FIG. 19.

The in parallel execution of W6/32 and anti-β2m—sera antibody sandwich assay is one of the best techniques for determining the presence and quantity of HLA positive antibodies. To detect antigen (sHLA)-specific allo-antibodies, the wells of microtiter plates are first coated with the specific (capture) antibody W6/32 or anti-β2m. Non-specific binding sites on the microtiter plates are blocked with a blocking agent, such as 3% BSA, followed by incubation with specific solutions containing excess amount of the sHLA antigen. Unbound antigen is washed out and the test sera are applied. To detect human IgG or IgM antibodies bound to the antigen, an anti-human IgG(M) antibody conjugated to HRP is added, followed by another incubation. Unbound conjugate is washed out, and a substrate, such as HRP-substrate, is added. After another incubation, the degree of substrate hydrolysis is measured. The amount of substrate hydrolyzed is proportional to the amount of antigen in the test solution.

The sensitivity of the assay depends on 4 factors: (1) the number of capture antibody; (2) the avidity of the capture antibody for the antigen; (3) the avidity of the sera for the antigen; and (4) the specific activity of the labeled second antibody.

The assay of the presently claimed invention is performed by first attaching sHLA molecules to a substrate, such as a solid support. The substrate may be any insoluble support to which the sHLA molecule can be bound, either directly or indirectly, which is readily separable from soluble material, and which is otherwise compatible with the overall methods of the present invention. The surface of such substrates may be solid or porous, and the substrates may have any shape that allows the substrate to function in accordance with the present invention. Examples of substrates that may be utilized in accordance with the present invention include, but are not limited to, microtiter plates, such as but not limited to ELISA plates; membranes, such as but not limited to, nitrocellulose membranes, PVDF membranes, nylon membranes, acetate derivatives, and combinations thereof; fiber matrix, SEPHAROSE® matrix, sugar matrix; plastic chips; glass chips; or any type of bead, such as but not limited to, LUMINEX® beads, DYNABEADS®, magnetic beads, flow-cytometry beads, and combinations thereof. The substrates are typically formed of glass, plastic or any other type of polymer, such as but not limited to PVC, polyvinyl propylene, polyethylene and the like, polysaccharides, nylon, nitrocellulose, and combinations thereof. Microtiter plates are especially convenient because a large number of assays can be carried out simultaneously, using small amounts of reagents and samples. Where separations are made by magnetism, the support generally includes paramagnetic components, preferably surrounded by plastic.

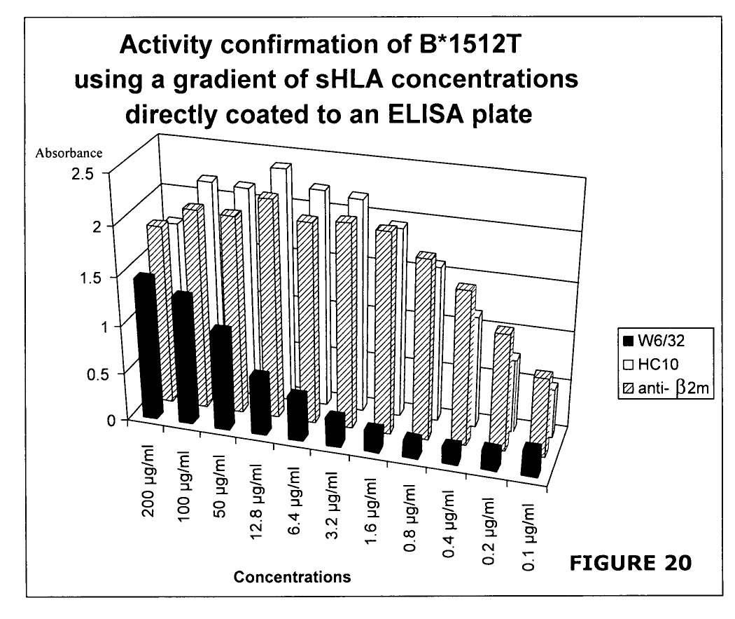

The sHLA molecules may be directly linked to the substrate, or the sHLA molecules may be indirectly linked to the substrate via an anchoring moiety. Direct attachment of sHLA molecules to the substrate may occur through several methods, including but not limited to, absorption, chemical coupling, and chemical linkage via a tail integrated by recombination to the sHLA molecule. Absorption involves non-specific binding of protein to any support. FIG. 20 illustrates confirmation of activity of sHLA B*1512T using a gradient of sHLA concentrations wherein the sHLA is directly coated to an ELISA plate. Concentrations of more than 12.8 μg/ml give a clear response with W6/32, recognizing only conformationally intact molecules. However, the procedure shown in FIG. 20 has not been optimized, as signals with HC10, an antibody recognizing heavy chain only, can be seen. In addition, anti-β2m recognizes both free and complexed β₂m molecules.

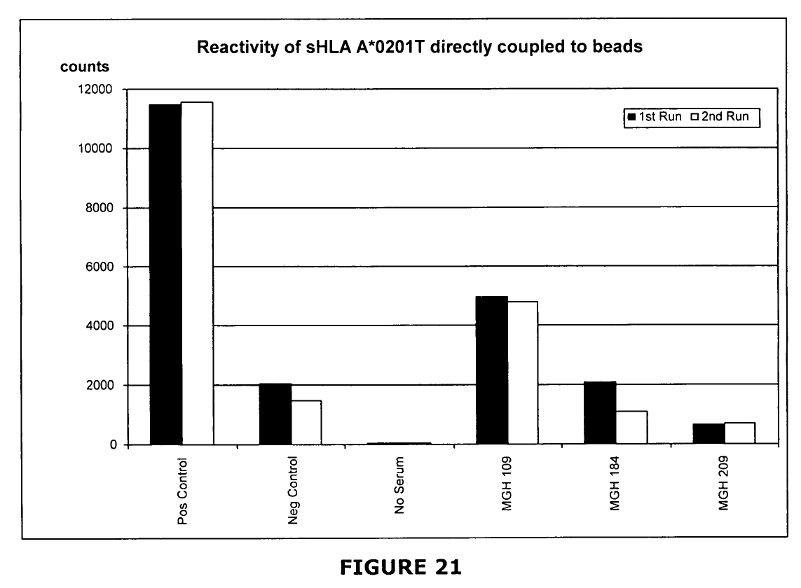

FIG. 21 illustrates reactivity of sHLA A*0201T directly coupled to beads via chemical coupling. sHLA was coupled via the EDC method to 1-ethyl-3-(3-dimethylaminopropyl) carbodiimide-HCl (EDC) activated beads. As seen in FIG. 21, sHLA A*0201T coupled to EDC beads was recognized correctly by human sera, while pooled, negative sera gave a negative response.

Optionally, the sHLA molecules may be indirectly linked to the substrate via an anchoring moiety. The terms “anchoring moiety” and “capture agent” may be used interchangeably herein. Examples of anchoring moieties that may be utilized in accordance with the present invention include, but are not limited to, antibodies, such as Pan-Class I and/or and allele-specific antibodies, as well as immune receptors with HLA binding affinity and lectins. The anchoring moiety may be bound to the surface of the substrate by any convenient means, depending upon the nature of the surface. The particular manner of binding is not crucial so long as it is compatible with the other reagents and overall methods of the invention. Where the anchoring moiety is antibody, it may be bound to the plates covalently or non-covalently, preferably non-covalently.

Preferred anchoring moieties include any antibody, whether polyclonal or monoclonal, that recognizes intact sHLA complexes, including Pan-Class I antibodies and/or allele-specific antibodies. Instead of whole or intact antibodies, one may use antibody fragments, e.g., Fab, F(ab′).sub.2, light or heavy chain fragments, etc.

Pan Class I antibodies such as but not limited to W6/32, anti-human β2m and TP25.99 can be utilized. W6/32 is one of the most common monoclonal antibodies (mAb) used to characterize human class I major histocompatibility complex (MHC) molecules. W6/32 is directed against monomorphic determinants on HLA-A, -B and -C HCs, which recognizes only mature complexed class I molecules. W6/32 recognizes a conformational epitope on the intact MHC molecule containing both beta2-microglobulin (beta2-m) and the heavy chain. Anti-human β2m is a polyclonal antibody that recognizes denatured as well as complexed β2m. The patterns of class I molecules precipitated with W6/32 and anti-β2m are usually indistinguishable [see Vasilov, 1983]. TP25.99 detects a determinant in the alpha-3 domain of HLA-A, B, and C. It is found on denatured HLA-B (in Western) as well as partially or fully folded HLA-A, B, and C. TP25.99 doesn't require a peptide or β2m, because it works with the alpha-3 domain which folds without peptide. This makes it also useful for HC determination.

A variety of monoclonal and polyclonal antibodies has also been reported to bind sHLA in a more specific manner, and are referred to herein as allele-specific antibodies. While such allele-specific antibodies may be utilized in accordance with the present invention, care should be taken in choosing one, as not all allele-specific antibodies are mono-specific and may therefore cross-react broadly.

Another type of anchoring moiety that may be utilized in accordance with the present invention is lectins. A hexosamine analysis of allele B*1512T revealed the presence of low amounts of glucosamine. This glucosamine content is only explainable by the presence of N-linked oligosaccharides attached to the HC. For human class I glycoproteins, asparagine 86 is the sole site for N-linked glycosylation.

A deduced sequence of the most dominant sialylated form of oligosaccharide found in all HLA class I preparations analyzed (Barber et al. 1996) is shown below. As seen, glucosamines are a main part of this structure, highly suggesting that our sHLA molecules are glycosylated.

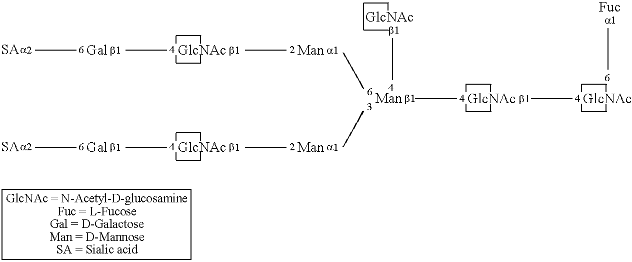

The oligosaccharide side chain usually has a molecular mass of 2700 to 3300 Da and is projecting away from the peptide-binding site. Although the asparagine 86 side chain to which the oligosaccharide is attached points away from the class I molecule, glycans exhibit considerable flexibility, and it is conceivable that one branch of the oligosaccharide could fold back, positioning it over to the protein. As such, this sugar can be used to indirectly link sHLA to any support by lectins.

In addition, immune molecules with alloantigen binding affinity such as CD4, CD8, and T cell receptors may also provide useful capture agents, either directly or through derivatives thereof. Lectins may be useful where the alloantigen can be selected by the presence of saccharides.

Before adding alloantigen samples, the non-specific binding sites on the substrate, i.e. those not occupied by sHLA linked directly or indirectly thereto, are blocked. Preferred blocking agents include non-interfering proteins such as bovine serum albumin, casein, gelatin, and the like.

Purified sHLA molecules are then added to separately assayable supports (for example, separate wells of a microtiter plate) containing substrate-bound anchoring moiety. One problem commonly encountered with the assays of the prior art involves detergents used in the solubilization of HLA. Detergents are a common problem in screening assays as they interfere with the test sera and cause high background values. By utilizing the purified sHLA molecules of the present invention, in which a defined buffer system has been utilized, background problems caused by detergents are not applicable as they are not used within the procedure to prepare sHLA molecules for antibody screening of the present claimed invention.

Generally from about 5 μg to about 10 μg of purified sHLA, diluted or otherwise, is sufficient for binding to the substrate directly and generally from about 8 ng to about 20 ng of purified sHLA, diluted or otherwise, is sufficient for binding to the anchoring moiety bound to the substrate. The incubation time should be sufficient for the sHLA molecules to bind the substrate or anchoring moiety. Generally, the incubation time will be in a range of from about 1 hr to about 2 hrs.

After each incubation step, the substrate is generally washed of non-bound components. Generally, a non-interfering buffered solution at an appropriate pH, generally 7-8, is used as a wash medium. Up to 5 to 10 washes may be employed, with sufficient volume to thoroughly wash non-specifically bound proteins present in the sample.

After washing, a biological sample possible containing at least one HLA-specific receptor is applied to detect a positive reaction. Samples, as used herein, include but are not limited to biological fluids such as blood, cerebrospinal fluid, tears, saliva, lymph, dialysis fluid and the like; organ or tissue culture derived fluids; and fluids extracted from physiological tissues. Also included in the term are derivatives and fractions of such fluids. A biological sample that will typically be utilized in the present invention is blood or derivatives thereof, such as serum or plasma. Such samples will generally be complex mixtures, where the concentration of specific receptor is low.

Particular receptors of interest are anti-HLA antibodies. The isotypes IgG and IgM will be found in blood, while IgA may be detected in secreted fluids, e.g., saliva, etc. Other receptors which may be indicative of an immune response are T-cell receptors. Of particular interest are anti-HLA antibodies found in the serum of transplant or prospective transplant patients. The volume, composition and concentration of the biological sample provides for measurable binding to individual sHLA already directly or indirectly bound to the substrate. The volume will generally be in a range of from about 30 μl to about 100 μl. The incubation time should be sufficient for the receptor to bind available bound sHLA molecules. Generally, the incubation time is in a range of from about 1 hr to about 2 hrs.

After the receptor has bound the sHLA, the substrate is generally again washed free from non-specifically bound proteins, essentially as described for prior washes. The presence of bound sHLA-specific receptor is detected with a labeled reagent, particularly anti-human antibodies, e.g. antisera. Examples of labels which permit direct measurement of receptor binding include radiolabels, such as ³H or ¹²⁵I, fluorescers, dyes, beads, chemilumninescers, colloidal particles, and the like. Examples of labels which permit indirect measurement of binding include enzymes where the substrate may provide for a colored or fluorescent product. In a preferred embodiment, the labeled reagents are antibodies, preferably labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

After non-specifically bound material has been cleared, the signal produced by the bound conjugate is detected by conventional means. Where an enzyme conjugate is used, an appropriate enzyme substrate is provided so a detectable product is formed. More specifically, where a peroxidase is the selected enzyme conjugate, a preferred substrate combination is H₂O₂ and o-phenylenediamine which yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For example, for the product of the substrate o-phenylenediamine, light absorbance at 490-495 nm is conveniently measured with a spectrophotometer.

Provided in Table A are typical detection systems that may be utilized with IgG or IgM recognizing human sera in accordance with the present invention.

TABLE A

| Label | Detection | System |
|---|---|---|
| HRP-labeled | OPD substrate | ELISA reader - OD490 |
| Biotinylated | ABC kit/OPD | ELISA reader - OD490 |
| Enzyme-labeled | substrate for enzyme | ELISA reader |
| | reaction producing | |
| | colored products | |
| Radioactive | Radiation | Scintillation counter |
| compound | | |
| Fluorescent | light emission after | Fluorescence reader |
| compound | excitation | |
| (any wavelength) | | |
| | | FACScan (Flow cytometry) |
| | | if bead support) |
| | | Fluorescence |
| | | Polarization if reaction |
| | | in solution |
| Dual fluorescent | | FRET |
| compounds | | |
| No label | Complex formation | Nephelometry (Laser) |
| gold particles | | Electron Microscope |

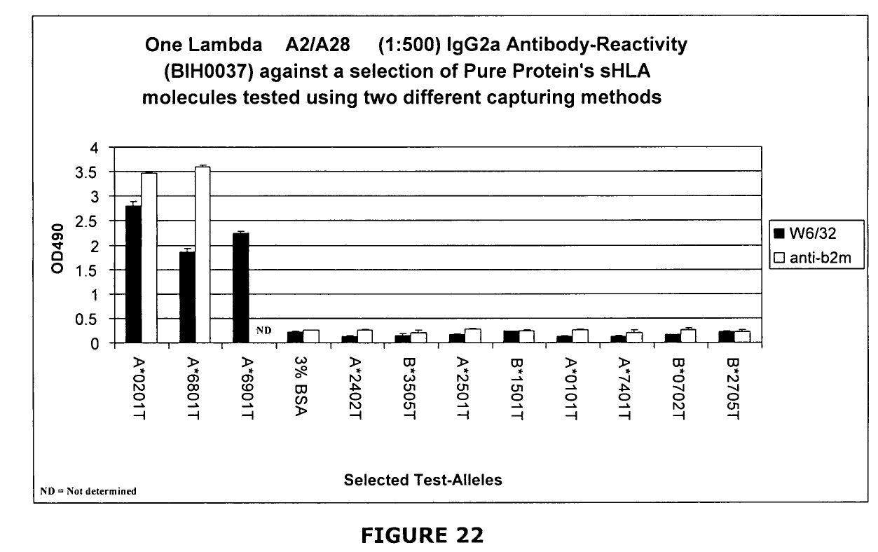

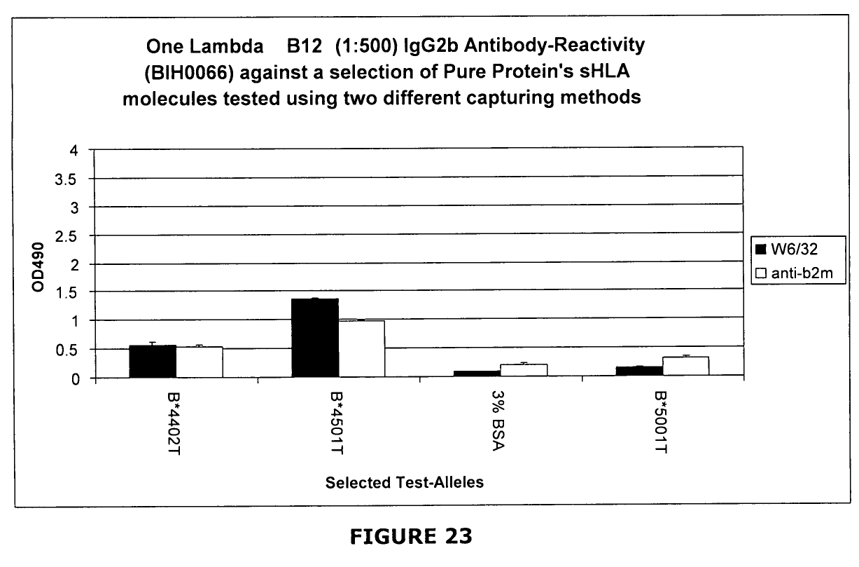

Preliminary studies to test the specificity of the sHLA of the present invention were conducted, as shown in FIGS. 22-23. In these tests, sHLA molecules were presented to commercially available monoclonal Ab with defined specificities through either a W6/32 or β2m capturing system. The graphs presented in FIGS. 22 and 23 demonstrate that commercially available antibodies recognize specific sHLA antigens in a correct manner, where non-related alleles did not respond.

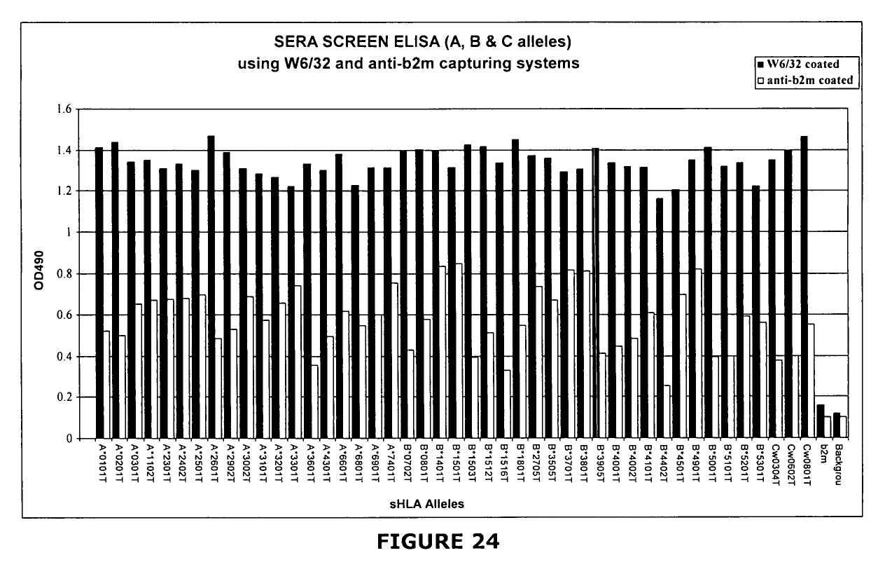

FIG. 24 illustrates an ELISA platform assay conducted to confirm the capability of W6/32 and anti-β2m to capture the produced sHLA molecules. As seen within this figure, both antibodies were able to capture sHLA and present them to the detection antibodies. Detection antibodies in case of the W6/32 capturing system was anti-β2m (HRP) and for the anti-β2m capturing system (W6/32-Biotin). At this point, sHLA concentrations were adjusted so that the capturing system is saturated for sera testing.

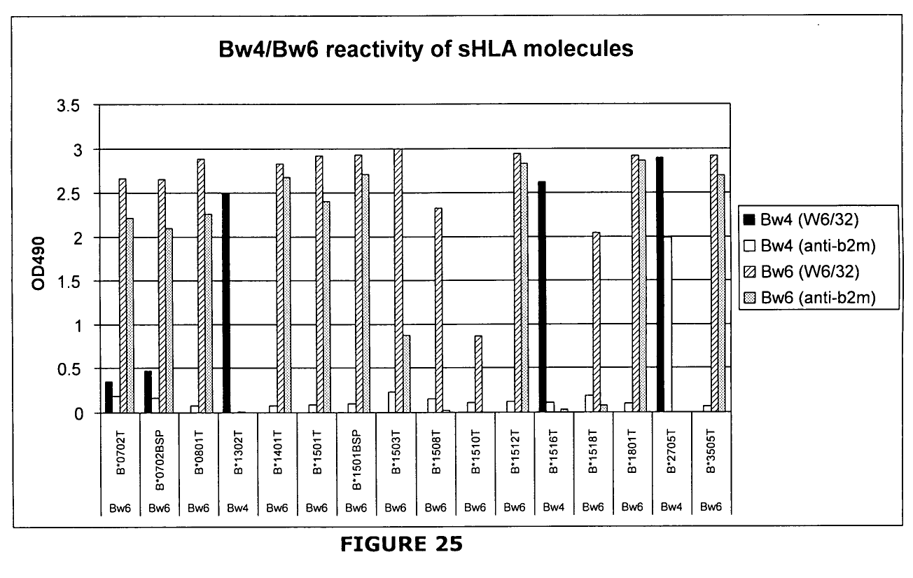

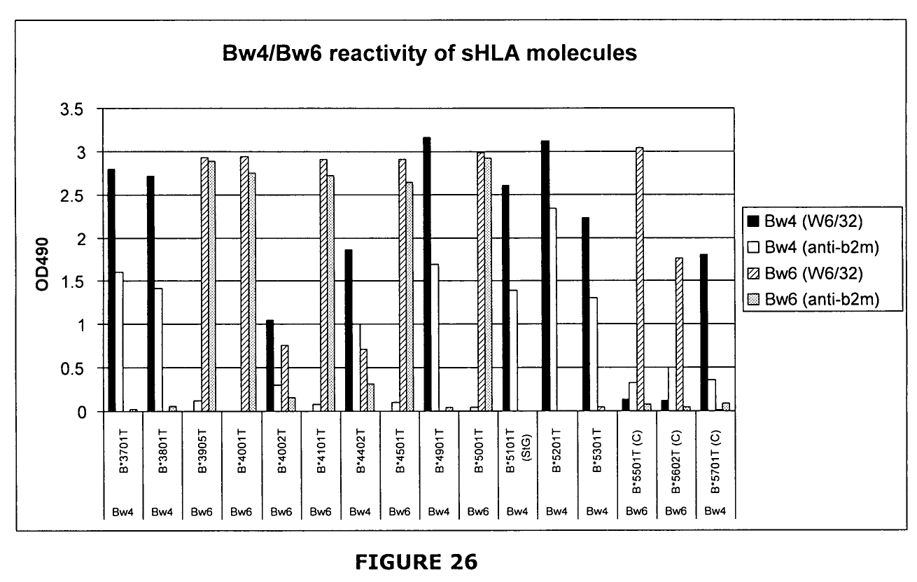

In order to demonstrate proper conformation of the sHLA class I proteins produced by the methods of the present invention, the alleles were tested using two different sandwich ELISA procedures. One procedure uses W6/32 as capturing antibody, whereas the other assay is coupled to anti-β2m as capturing antibody. In FIGS. 25 and 26, Bw6 and Bw4 Abs were tested. Each Ab is known to recognize a conserved epitope on B alleles. However, Bw6 positive alleles are Bw4 negative and vice versa.

These tests confirmed as expected that all alleles harbor either the Bw6 or the Bw4 epitope. All results agree with the current nomenclature of Bw4/Bw6 sorting.

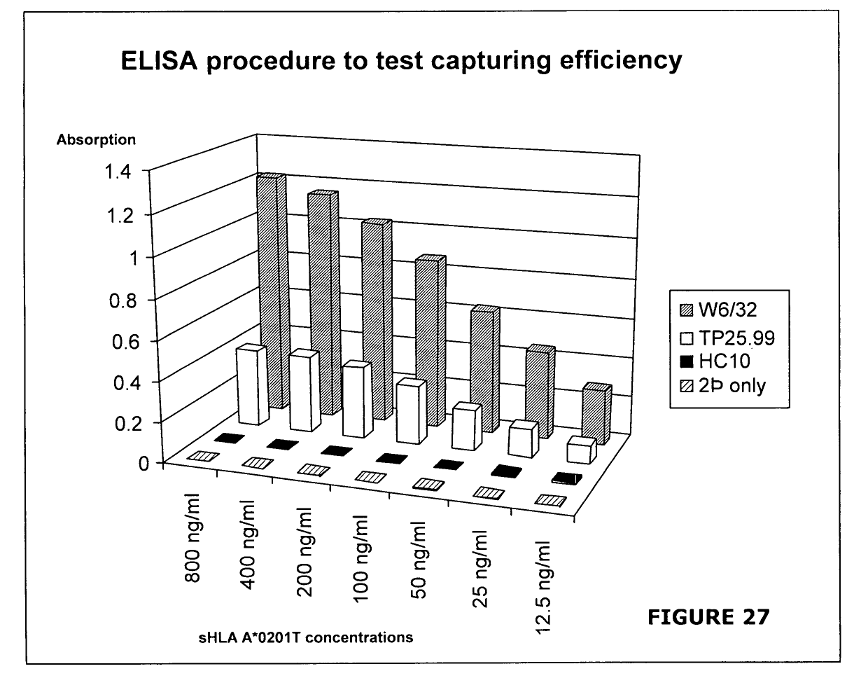

In FIG. 27, the efficiency to produce a signal was tested between W6/32 and the Sangstat antibody TP25.99. This titration experiment clearly demonstrates that the capability of TP25.99 to capture sHLA is much less than the W6/32, as equal amounts of Ab was used within this test series. The antibody HC10 did not produce any signal, confirming the absence of free heavy chain. Background levels were minimal as seen by incubation of 2° Ab only.

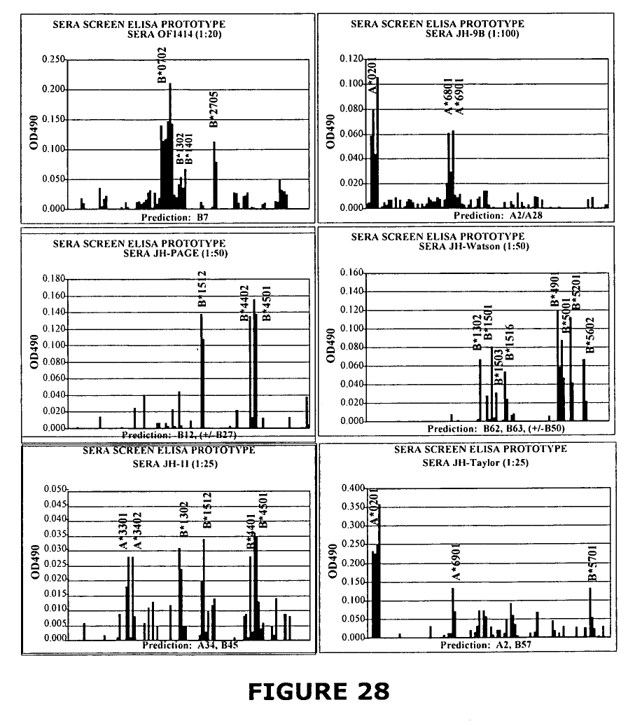

Using the platform assay exactly as described herein, several test sera were run and characterized (FIG. 28). All sera seen showed an exact match to the predictions obtained by established HLA testing labs such as John Hopkins or Ochsner Transplantation Center. Furthermore, the tests give exquisite conclusions on predictions which were not conclusive, pointing to a much higher sensitivity of the assay of the present invention as compared to other commercially available tests.

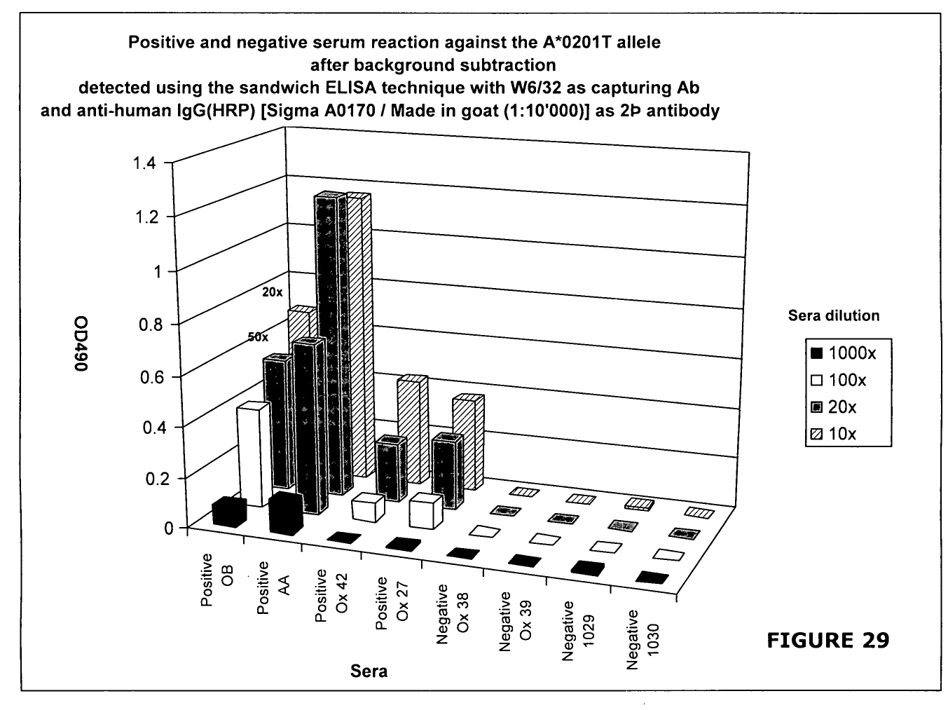

Optimization of the 2° Ab response was performed by testing positive and negative characterized sera in both W6/32 and anti-β2m systems using a gradient of the test sera. The results shown in FIG. 29 clearly demonstrate that signals for positive sera are much higher than for negative sera, and a distinguishable response could be detected up to 1000× dilution of Sera AA and OB. In addition, the secondary Ab does not produce high background levels usually observed with other 2° Ab which cross-react with rabbit or mouse IgG.

### Materials and Methods

Material and Methods involved in the production, isolation and purification of functionally active, individual soluble HLA molecules are described in detail in U.S. Ser. No. 10/337,161, previously incorporated herein by reference, and such materials and methods are expressly incorporated herein by reference in their entirety.

Sera Screening using the W6/32 and anti-β2m HLA sandwich ELISAs are designed using an ELISA protocol template in an 8×8 format with 64 test wells. The plate contains: 50 different alleles (20 A*'s/27 B*'s/3 Cw*'s), 4 tailed molecules (A*0201VLDL/B*0702BSP/B*0702His/B*1501BSP), 1 b2-microglobulin control and 9 blanks (3% BSA). Polystyrene assay plates are used in the procedure (Immuno Module Maxisorp F8 framed (Nunc)). In the first step, plates were coated with the capturing antibodies W6/32 (8.0 μg/ml) (Pure Protein) and anti-β2m (10.0 μg/ml) (DAKO) in Tris buffered saline (TBS); pH 8.5. After incubation at 4° C. overnight, plates were washed 10 times with Wash Buffer (PBS containing 0.05% Tween-20) using a multi-channel ELISA washer. After the coating antibodies were bound, the remaining sites on the plate were blocked with 3% BSA in PBS and incubated overnight at 4° C. sHLA molecules were prepared at a concentration of 300 ng/ml, which is over the dynamic range of binding and sufficient to saturate the capacity of the capturing antibodies. After washing, single molecules were loaded onto the plate and incubated for 1 hour. After incubation of sHLA, unbound antigen was washed away and test sera or antibody was added for another hour. Finally, after washing non-reactive sera/antibody from the plates, a secondary antibody (goat anti-human IgG (Sigma A0170) (4.6 mg/ml) was used at a ratio of 1:10,000 in 3% BSA in PBS for human sera. For monoclonal antibodies as test compounds, commercial goat anti-mouse IgG was used. After incubation at 20 minutes to 1 hour followed by a last wash, OPD (o-Phenylenediamine) peroxidase substrate (Sigma, P6787; 2 mg/tablet) was used to visualize positive wells. The OPD reaction was finally stopped with 3 N H₂SO₄ and read at 492 nm.

The platform and antigenic integrity assays (FIGS. 85-87) were conducted as described previously, except that after incubation of sHLA, unbound antigen was washed away and test antibody (biotinylated) was added for another hour. Finally, after washing non-reactive antibody from the plates, a biotin detection system (ABC, Vectastain) was used (step 5). In the case of anti-β2m detection, the ABC step was skipped. After incubation for 20 minutes and last wash, OP peroxidase substrate was used to visualize positive wells. The OPD reaction was finally stopped as described above and read at 492 nm.

Thus, in accordance with the present invention, there has been provided herein methods for the detection of anti-HLA antibodies in a sample utilizing purified, functionally active, individual soluble HLA molecules that fully satisfies the objectives and advantages set forth herein above. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference in their entirety as though set forth herein particular.
Bjorkman, P. J. and P. Parham, Structure, function, and diversity of class I major histocompatibility complex molecules. Annu Rev Biochem, 1990. 59: p. 253-88.
Bjorkman, P. J., Saper, M. A., Samraoui, B., Bennett, W. S., Strominger, J. L. and Wiley, D. C. (1987), “Structure of the human class I histocompatibility antigen, HLA-A2”, Nature 329: 506-12.
   Borrow, P., Lewicki, H., Hahn, B. H., Shaw, G. M. and Oldstone, M. B. (1994), “Virus-specific CD8+ cytotoxic T-lymphocyte activity associated with control of viremia in primary human immunodeficiency virus type 1 infection”, J Virol 68: 6103-10.
Collins, E. J., et al., The three-dimensional structure of a class I major histocompatibility complex molecule missing the alpha 3 domain of the heavy chain. Proc Natl Acad Sci USA, 1995. 92(4): p. 1218-21.
Cresswell, P., M. J. Turner, and J. L. Strominger, Papain-solubilized HL-A antigens from cultured human lymphocytes contain two peptide fragments. Proc Natl Acad Sci USA, 1973. 70(5): p. 1603-7.
Cresswell, P., et al., Papain-solubilized HL-A antigens. Chromatographic and electrophoretic studies of the two subunits from different specificities. J Biol Chem, 1974. 249(9): p. 2828-32.
Dedier, S., Reinelt, S., Rion, S., Folkers, G. and Rognan, D. (2001), “Use of fluorescence polarization to monitor MHC-peptide interactions in solution”, J Immunol Methods 255: 57-66.
Garboczi, D. N., Hung, D. T. and Wiley, D. C. (1992), “HLA-A2-peptide complexes: refolding and crystallization of molecules expressed in Escherichia coli and complexed with single antigenic peptides”, Proc Natl Acad Sci USA 89: 3429-33.
Harty, J. T. and Bevan, M. J. (1992), “CD8+ T cells specific for a single nonamer epitope of Listeria monocytogenes are protective in vivo”, J Exp Med 175: 1531-8.
Heslop, H. E., Ng, C. Y., Li, C., Smith, C. A., Loftin, S. K., Krance, R. A., Brenner, M. K. and Rooney, C. M. (1996), “Long-term restoration of immunity against Epstein-Barr virus infection by adoptive transfer of gene-modified virus-specific T lymphocytes”, Nat Med 2: 551-5.
Laemmli, U. K et al., Cleavage of structural proteins during the assembly of the head of bacteriophage T4. Nature, 1970, 227, p. 680-685.
Levy, F. and Kvist, S. (1990), “Co-expression of the human HLA-B27 class I antigen and the E3/19K protein of adenovirus-2 in insect cells using a baculovirus vector”, Int Immunol 2: 995-1002.
Melief, C. J. (1992), “Tumor eradication by adoptive transfer of cytotoxic T lymphocytes”, Adv Cancer Res 58: 143-75.
Melief, C. J. and Kast, W. M. (1992), “Lessons from T cell responses to virus induced tumours for cancer eradication in general”, Cancer Surv 13: 81-99.
Mills, K. H., Nixon, D. F. and McMichael, A. J. (1989), “T-cell strategies in AIDS vaccines: MHC-restricted T-cell responses to HIV proteins”, Aids 3:
Parker, K. C., Carreno, B. M., Sestak, L., Utz, U., Biddison, W. E. and Coligan, J. E. (1992), “Peptide binding to HLA-A2 and HLA-B27 isolated from Escherichia coli. Reconstitution of HLA-A2 and HLA-B27 heavy chain/beta 2-microglobulin complexes requires specific peptides”, 3 Biol Chem 267: 5451-9.
Peterson, P. A., Rask, L. and Lindblom, J. B. (1974), “Highly purified papain-solubilized HL-A antigens contain beta2-microglobulin”, Proc Natl Acad Sci USA 71: 35-9.
Prilliman, K., Lindsey, M., Zuo, Y., Jackson, K. W., Zhang, Y. and Hildebrand, W. (1997), “Large-scale production of class I bound peptides: assigning a signature to HLA-B*1501”, Immunogenetics 45: 379-85.
Prilliman, K. R., Jackson, K. W., Lindsey, M., Wang, J., Crawford, D. and Hildebrand, W. H. (1999), “HLA-B15 peptide ligands are preferentially anchored at their C termini”, J Immunol 162: 7277-84.
Prilliman, K. R., Lindsey, M., Wang, J., Jackson, K. W. and Hildebrand, W. H. (1999), “Peptide motif of the class I molecule HLA-B*1503”, Immunogenetics 49: 144-6.
Sakaguchi, T., Ibe, M., Miwa, K., Kaneko, Y., Yokota, S., Tanaka, K. and Takiguchi, M. (1997), “Binding of 8-mer to 11-mer peptides carrying the anchor residues to slow assembling HLA class I molecules (HLA-B*5101)”, Immunogenetics 45: 259-65.
Schmitz, J. E., Kuroda, M. J., Santra, S., Sasseville, V. G., Simon, M. A., et al. (1999), “Control of viremia in simian immunodeficiency virus infection by CD8+ lymphocytes”, Science 283: 857-60.
Schumacher, T. N., De Bruijn, M. L., Vernie, L. N., Kast, W. M., Melief, C. J., Neefjes, J. J. and Ploegh, H. L. (1991), “Peptide selection by MHC class I molecules”, Nature 350: 703-6.
Silver, M. L., Parker, K. C. and Wiley, D. C. (1991), “Reconstitution by MHC-restricted peptides of HLA-A2 heavy chain with beta 2-microglobulin, in vitro”, Nature 350: 619-22.
Tanigaki, N., Katagiri, M., Nakamuro, K., Kreiter, V. P. and Pressman, D. (1974), “Common antigenic structures of HL-A antigens. II. Small fragments derived from papain-solubilized HL-A antigen molecules”, Immunology 26: 155-68.
Tanigaki, N. and Pressman, D. (1974), “The basic structure and the antigenic characteristics of HL-A antigens”, Transplant Rev 21: 15-34.
Townsend, A. and Bodmer, H. (1989), “Antigen recognition by class I-restricted T lymphocytes”, Annu Rev Immunol 7: 601-24.

What is claimed is:

1. A method for removing anti-MHC antibodies from a biological sample, the method comprising the steps of:
providing a substrate;
providing a pool of functionally active, recombinantly produced, truncated individual soluble MHC trimolecular complexes, each trimolecular complex comprising a recombinant, soluble MHC heavy chain allele, beta-2-microglobulin, and endogenously loaded peptide, the functionally active, recombinantly produced, truncated individual soluble MHC trimolecular complexes being purified substantially away from other proteins such that the individual soluble MHC trimolecular complexes maintain the physical, functional and antigenic integrity of the native MHC trimolecular complex, wherein the individual soluble MHC trimolecular complexes are produced by a method comprising the steps of:
isolating mRNA from a source, wherein the mRNA encodes at least one MHC heavy chain allele;
reverse transcribing the mRNA to obtain cDNA;
identifying an individual MHC heavy chain allele in the cDNA;
PCR amplifying the individual MHC heavy chain allele in a locus-specific manner to produce a PCR product having the coding regions encoding cytoplasmic and transmembrane domains of the individual MHC heavy chain allele removed such that the PCR product encodes a truncated, soluble form of the individual MHC heavy chain molecule;
cloning the PCR product into a mammalian expression vector, thereby forming a construct that encodes the individual soluble MHC heavy chain molecule;
transfecting a mammalian cell line with the construct to provide a mammalian cell line expressing a construct that encodes a recombinant, individual soluble MHC heavy chain molecule, wherein the mammalian cell line is able to naturally process proteins into peptide ligands for loading into antigen binding grooves of MHC molecules;
culturing the mammalian cell line under conditions which allow for expression of the recombinant individual soluble MHC heavy chain molecule from the construct, such conditions also allowing for endogenous loading of a peptide ligand into the antigen binding groove of each individual soluble MHC heavy chain molecule in the presence of beta-2-microglobulin to form the individual soluble MHC trimolecular complexes prior to secretion of the individual soluble MHC trimolecular complexes from the cell; and
purifying the individual, soluble MHC trimolecular complexes substantially away from other proteins, wherein the individual soluble MHC trimolecular complexes maintain the physical, functional and antigenic integrity of the native MHC trimolecular complex;
linking at least one soluble MHC trimolecular complex to the substrate, wherein the at least one soluble MHC trimolecular complex is directly or indirectly linked to the substrate, and wherein the at least one soluble MHC trimolecular complex linked to the substrate retains the physical, functional and antigenic integrity of the native MHC trimolecular complex;
providing a biological sample; and
reacting the biological sample with the substrate having the at least one MHC trimolecular complex linked thereto, whereby antibodies specific for the at least one MHC trimolecular complex linked to the substrate are removed from the biological sample.

2. The method of claim 1 wherein, in the step of providing a substrate, the substrate is a solid support.

3. The method of claim 2, wherein the solid support is selected from the group consisting of a column, a well, a bead, a membrane, an ELISA plate, and a matrix.

4. The method of claim 1 wherein, in the step of linking at least one soluble MHC trimolecular complex to the substrate, the at least one soluble MHC trimolecular complex is indirectly attached to the substrate via an anchoring moiety.

5. The method of claim 4, wherein the anchoring moiety comprises an antibody to the functionally active, individual soluble MHC trimolecular complex.

6. The method of claim 5, wherein the antibody is selected from the group consisting of W6/32, anti-beta 2m, pan-Class I or allele-specific antibodies and combinations thereof.

7. The method of claim 4, wherein the anchoring moiety comprises a tail or tag attached to the functionally active, individual soluble MHC trimolecular complex, and the substrate is further defined as comprising an affinity reagent to which the tail or tag binds.

8. The method of claim 7, wherein the tail or tag is a histidine tag, and the affinity reagent is selected from the group consisting of nickel, copper and combinations thereof.

9. The method of claim 7, wherein the tail or tag is a biotinylation signal peptide, and the affinity reagent is avidin or streptavidin.

10. The method of claim 7, wherein the tail or tag is a VLDLr or FLAG tail, and the affinity reagent is an antibody that recognizes the VLDLr or FLAG tail.

11. The method of claim 1 wherein, in the step of providing a pool of functionally active, recombinantly produced, truncated individual soluble MHC trimolecular complexes, the pool of functionally active, recombinantly produced, truncated individual soluble MHC trimolecular complexes are Class I or Class II MHC trimolecular complexes.

12. The method of claim 1 wherein, in the step of isolating mRNA from a source, the source is selected from the group consisting of mammalian DNA and an immortalized cell line.

13. The method of claim 1 wherein, in the step of cloning the PCR product into a mammalian expression vector, the mammalian expression vector contains a promoter that facilitates increased expression of the truncated PCR product.

14. The method of claim 1 wherein, in the step of PCR amplifying the individual MHC heavy chain allele, a primer utilized in the PCR amplification includes a sequence encoding a tail such that the soluble MHC heavy chain molecule encoded by the truncated PCR product contains a tail attached thereto that facilitates in purification of the soluble MHC trimolecular complexes produced there from or facilitates in direct binding of the soluble MHC trimolecular complexes to the substrate.

15. The method of claim 1 wherein, in the step of PCR amplifying the individual MHC heavy chain allele, a 3′ primer utilized in the PCR amplification includes a stop codon incorporated therein.

16. The method of claim 1 wherein, in the step of purifying the individual, soluble MHC trimolecular complexes substantially away from other proteins, the functionally active, individual soluble MHC trimolecular complexes are purified by affinity chromatography and fractionation.

17. The method of claim 16 wherein the affinity chromatography utilizes a reagent selected from the group consisting of W6/32 antibodies, anti-β2m antibodies, pan-Class I antibodies or allele-specific antibodies, and combinations thereof.

18. The method of claim 1 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.

19. A method for removing anti-MHC antibodies from a biological sample, the method comprising the steps of:
providing a substrate;
providing a pool of functionally active, recombinantly produced, truncated individual soluble MHC trimolecular complexes, each trimolecular complex comprising a recombinant, soluble MHC heavy chain allele, beta-2-microglobulin, and endogenously loaded peptide, the functionally active, recombinantly produced, truncated individual soluble MHC trimolecular complexes being purified substantially away from other proteins such that the individual soluble MHC trimolecular complexes maintain the physical, functional and antigenic integrity of the native MHC trimolecular complex, wherein the individual soluble MHC trimolecular complexes are produced by a method comprising the steps of:
obtaining gDNA, wherein the gDNA encodes at least one MHC heavy chain allele;
identifying an individual MHC heavy chain allele in the gDNA;
PCR amplifying the individual MHC heavy chain allele in a locus-specific manner to produce a PCR product having the coding regions encoding cytoplasmic and transmembrane domains of the individual MHC heavy chain allele removed such that the PCR product encodes a truncated, soluble form of the individual MHC heavy chain molecule;
cloning the PCR product into a mammalian expression vector, thereby forming a construct that encodes the individual soluble MHC heavy chain molecule;
transfecting a mammalian cell line with the construct to provide a mammalian cell line expressing a construct that encodes a recombinant, individual soluble MHC heavy chain molecule, wherein the mammalian cell line is able to naturally process proteins into peptide ligands for loading into antigen binding grooves of MHC molecules;
culturing the mammalian cell line under conditions which allow for expression of the recombinant individual soluble MHC heavy chain molecule from the construct, such conditions also allowing for endogenous loading of a peptide ligand into the antigen binding groove of each individual soluble MHC heavy chain molecule in the presence of beta-2-microglobulin to form the individual soluble MHC trimolecular complexes prior to secretion of the individual soluble MHC trimolecular complexes from the cell; and
purifying the individual, soluble MHC trimolecular complexes substantially away from other proteins, wherein the individual soluble MHC trimolecular complexes maintain the physical, functional and antigenic integrity of the native MHC trimolecular complex;
linking at least one soluble MHC trimolecular complex to the substrate, wherein the at least one soluble MHC trimolecular complex is directly or indirectly linked to the substrate, and wherein the at least one soluble MHC trimolecular complex linked to the substrate retains the physical, functional and antigenic integrity of the native MHC trimolecular complex;
providing a biological sample; and
reacting the biological sample with the substrate having the at least one MHC trimolecular complex linked thereto, whereby antibodies specific for the at least one MHC trimolecular complex linked to the substrate are removed from the biological sample.

20. The method of claim 19 wherein, in the step of providing a substrate, the substrate is a solid support.

21. The method of claim 20, wherein the solid support is selected from the group consisting of a column, a well, a bead, a membrane, an ELISA plate, and a matrix.

22. The method of claim 19 wherein, in the step of linking at least one soluble MHC trimolecular complex to a substrate, the at least one soluble MHC trimolecular complex is indirectly attached to the substrate via an anchoring moiety.

23. The method of claim 22, wherein the anchoring moiety comprises an antibody to the functionally active, individual soluble MHC trimolecular complex.

24. The method of claim 23, wherein the antibody is selected from the group consisting of W6/32, anti-beta 2m, pan-Class I or allele-specific antibodies and combinations thereof.

25. The method of claim 22, wherein the anchoring moiety comprises a tail or tag attached to the functionally active, individual soluble MHC trimolecular complex, and the substrate is further defined as comprising an affinity reagent to which the tail or tag binds.

26. The method of claim 25, wherein the tail or tag is a histidine tag, and the affinity reagent is selected from the group consisting of nickel, copper and combinations thereof.

27. The method of claim 25, wherein the tail or tag is a biotinylation signal peptide, and the affinity reagent is avidin or streptavidin.

28. The method of claim 25, wherein the tail or tag is a VLDLr or FLAG tail, and the affinity reagent is an antibody that recognizes the VLDLr or FLAG tail.

29. The method of claim 19 wherein, in the step of providing a pool of functionally active, recombinantly produced, truncated individual soluble MHC trimolecular complexes, the pool of functionally active, recombinantly produced, truncated individual soluble MHC trimolecular complexes are Class I or Class II MHC trimolecular complexes.

30. The method of claim 19 wherein, in the step of cloning the PCR product into a mammalian expression vector, the mammalian expression vector contains a promoter that facilitates increased expression of the truncated PCR product.

31. The method of claim 19 wherein, in the step of PCR amplifying the individual MHC heavy chain allele, a primer utilized in the PCR amplification includes a sequence encoding a tail such that the soluble MHC heavy chain molecule encoded by the truncated PCR product contains a tail attached thereto that facilitates in purification of the soluble MHC trimolecular complexes produced there from or facilitates in direct binding of the soluble MHC trimolecular complexes to the substrate.

32. The method of claim 19 wherein, in the step of PCR amplifying the individual MHC heavy chain allele, a 3′ primer utilized in the PCR amplification includes a stop codon incorporated therein.

33. The method of claim 19 wherein, in the step of purifying the individual, soluble MHC trimolecular complexes substantially away from other proteins, the functionally active, individual soluble MHC trimolecular complexes are purified by affinity chromatography and fractionation.

34. The method of claim 33, wherein the affinity chromatography utilizes a reagent selected from the group consisting of W6/32 antibodies, anti-b2m antibodies, pan-Class I antibodies or allele-specific antibodies, and combinations thereof.

35. The method of claim 19 wherein, in the step of providing a biological sample, the biological sample is selected from the group consisting of serum, tissue, blood, cerebrospinal fluid, tears, saliva, lymph, dialysis fluid, organ or tissue culture derived fluids, fluids extracted from physiological tissues, and combinations thereof.


SEQUENCE LISTING (8 sequences)

SEQ ID NO: 1 - protein, 10 aa, Homo sapiens
GSHSMRYFFT

SEQ ID NO: 2 - protein, 9 aa, Homo sapiens
IQHTMKIFY

SEQ ID NO: 3 - protein, 10 aa, Homo sapiens
GSRSPRYQVT

SEQ ID NO: 4 - protein, 10 aa, Homo sapiens
IQRTPKIQVY

SEQ ID NO: 5 - protein, 9 aa, Homo sapiens
  misc_feature (1)..(1): Xaa can be any naturally occurring amino acid
XQKDIXXXY

SEQ ID NO: 6 - protein, 9 aa, Homo sapiens
  misc_feature (1)..(1): Xaa can be any naturally occurring amino acid
XLPPVXXXF

SEQ ID NO: 7 - protein, 9 aa, Homo sapiens
  misc_feature (1)..(2): Xaa can be any naturally occurring amino acid
XXKDIXXXY

SEQ ID NO: 8 - protein, 9 aa, Homo sapiens
  misc_feature (1)..(2): Xaa can be any naturally occurring amino acid
XXPPXXXXF